(19) United States
(12) Patent Application Publication
(10) Pub. No.: US 2025/0197435 A1
(43) Pub. Date: Jun. 19, 2025

(54) PHOTOCATALYTIC VALORIZATION OF LIGNIN

(71) Applicant: The Trustees of Boston College, Chestnut Hill, MA (US)

(72) Inventors: Hongyan Wang, Brighton, MA (US); Gavin J. Giardino, Brighton, MA (US); Jia Niu, Lexington, MA (US); Dunwei Wang, Newton, MA (US)

(21) Appl. No.: 18/977,084

(22) Filed: Dec. 11, 2024

Related U.S. Application Data
(60) Provisional application No. 63/610,385, filed on Dec. 14, 2023.

(51) Int. Cl. C07G 1/00; B01J 23/30; C07C 51/31

(52) U.S. Cl. CPC C07G 1/00; B01J 23/30; C07C 51/313

(57) ABSTRACT

A method for selective partial depolymerization of natural lignin involves obtaining a natural lignin source and performing photocatalytic activation of the β-O-4 bond using a system with tetrabutylammonium decatungstate (TBADT) as a catalyst and ultraviolet light. The method includes adding an exogenous electron mediator/scavenger system to influence the cleavage or oxidation of the β-O-4 bond. The exogenous electron mediator enhances oligomer yield while reducing carbonyl (C═O) groups, whereas the exogenous electron scavenger increases carbonyl (C═O) groups with minimal impact on oligomer yield. The oligomer yield is calculated as the weight of the oligomer divided by the weight of the natural lignin source.

### CROSS-REFERENCE TO RELATED APPLICATIONS

This application claims the benefit of priority to U.S. Provisional Patent No. 63/610,385, filed on Dec. 14, 2023, the entirety of which is incorporated by reference as if fully reproduced and set forth herein in its entirety.

### STATEMENT REGARDING FEDERALLY SPONSORED RESEARCH OR DEVELOPMENT

This invention was made with government support under CBET 2037844, CHE 2117246, and CHE 1944512 awarded by the National Science Foundation. The government has certain rights in the invention.

### FIELD OF THE INVENTION

This invention relates to processes of converting lignin, a byproduct of lignocellulose pretreatment, into valuable products by a controlled lignin valorization.

### BACKGROUND OF THE INVENTION

Lignin is a complex organic polymer found in the cell walls of plants, making it one of the most abundant natural polymers on Earth. It plays a crucial role in providing structural support, water transport, and resistance to microbial attack in plants. However, its complex and irregular structure has historically made it challenging to break down and utilize effectively. As a result, lignin is often considered a waste product in industries such as paper manufacturing, where it is typically burned for energy rather than being used as a raw material for higher-value products. While it is recognized that lignin contains many potentially high-value components, the valorization of lignin presents severe technical and industrial/chemical challenges.

The challenges associated with lignin valorization are not only technical but also economic and regulatory. The development of cost-effective processes that can compete with traditional fossil-fuel-based products is essential for the widespread adoption of lignin-derived materials. Additionally, regulatory frameworks that support the use of renewable resources and incentivize sustainable practices will play a crucial role in driving the lignin economy forward.

Collaboration between academia, industry, and government is vital to overcoming these challenges and unlocking the full potential of lignin. By fostering partnerships and sharing knowledge, stakeholders can accelerate the development of innovative solutions and create a more sustainable future. As research continues to advance, the vision of a lignin-based economy, where this abundant natural resource is fully utilized, becomes increasingly attainable.

Lignin represents a promising opportunity for the development of sustainable materials and chemicals. Its abundance, coupled with the growing demand for renewable resources, makes it a key focus of research and innovation. By addressing the technical, economic, and regulatory challenges associated with lignin valorization, it is possible to pave the way for a more sustainable and circular economy, where lignin is transformed from a waste product into a valuable resource.

The depolymerization of lignin into smaller, more manageable molecules has been a significant area of research, as it holds the potential to transform lignin from a low-value byproduct into a valuable resource for producing bio-based chemicals and materials. Traditional methods of lignin depolymerization often require harsh chemical conditions and result in low yields of useful products. There is a growing need for more efficient and selective methods to break down lignin into oligomers that can be further processed into recyclable polymers. This need is driven by the increasing demand for sustainable and renewable materials that can replace fossil-fuel-derived products, as well as the desire to improve the economic viability of lignin valorization processes.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

Approaches to the depolymerization of lignin, a complex and irregular biopolymer found in the cell walls of plants, have primarily focused on thermal, chemical, and biological methods. Thermal methods, such as pyrolysis, involve the application of high temperatures to break down lignin into smaller molecules. However, these methods often result in a broad distribution of products and require significant energy input, making them less efficient and environmentally friendly. Chemical methods, including acid or base catalysis, have been employed to cleave lignin bonds, but these approaches can lead to harsh reaction conditions and the formation of undesirable by-products. Biological methods, utilizing enzymes or microorganisms, offer a more environmentally benign alternative, but they typically suffer from slow reaction rates and limited substrate specificity.

In the technology disclosed herein, catalytic and photocatalytic methods have emerged as a promising alternative for lignin depolymerization. These methods leverage the use of light to activate catalysts that can selectively cleave specific bonds within the lignin structure. Photocatalysts such as titanium dioxide (TiO2) and other metal oxides have been explored for this purpose. While these systems can offer improved selectivity and milder reaction conditions compared to traditional methods, they often require the use of high-energy UV light and can suffer from low efficiency and limited scalability. Additionally, the challenge of achieving selective cleavage of specific lignin bonds, such as the β-O-4 bond, remains a significant hurdle in the development of effective photocatalytic depolymerization processes.

Another approach involves the use of electron mediators and scavengers to enhance the efficiency of lignin depolymerization. Electron mediators can facilitate electron transfer processes, potentially improving the selectivity and yield of desired products. Electron scavengers, on the other hand, can help control the oxidation state of the reaction environment, influencing the types of products formed. Despite the potential benefits, the integration of these components into a cohesive and efficient depolymerization system has proven challenging. The complexity of lignin's structure and the need for precise control over reaction conditions to achieve desired product distributions continue to pose significant obstacles.

However, none of the previous approaches have provided a comprehensive solution that combines the features and details described in this disclosure.

Lignin's potential as a renewable resource is vast, given its abundance and the increasing interest in sustainable practices. The global shift towards a circular economy, where waste is minimized and resources are reused, has put lignin in the spotlight as a potential key player. Researchers are exploring various innovative approaches to lignin valorization, including biological, chemical, and thermal methods. Each of these methods offers unique advantages and challenges, and ongoing research aims to optimize these processes to maximize yield and efficiency.

Biological methods of lignin depolymerization involve the use of microorganisms or enzymes to break down lignin into smaller components. These methods are often considered more environmentally friendly compared to chemical processes, as they typically operate under milder conditions and produce fewer byproducts. However, the complexity of lignin's structure poses a significant challenge for biological degradation, and much research is focused on identifying and engineering microorganisms and enzymes that can effectively target lignin's bonds.

Chemical methods, on the other hand, involve the use of catalysts and solvents to facilitate the breakdown of lignin. Advances in catalysis have led to the development of more selective and efficient processes, allowing for the production of specific lignin-derived compounds. These compounds can serve as precursors for a wide range of applications, including the production of adhesives, resins, and even carbon fibers. The development of green chemistry approaches, which aim to reduce the environmental impact of chemical processes, is also playing a crucial role in advancing lignin valorization.

The integration of lignin valorization into existing industrial processes is another area of active research. By developing technologies that can be seamlessly incorporated into current manufacturing systems, the economic feasibility of lignin utilization can be significantly enhanced. This integration could lead to the development of biorefineries, where lignin and other biomass components are processed into a range of valuable products, thereby maximizing resource efficiency and minimizing waste.

In addition to its potential as a source of bio-based chemicals, lignin is also being explored for its unique properties in material science. Its natural UV resistance, antioxidant properties, and ability to form films and fibers make it an attractive candidate for the development of new materials. Researchers are investigating the use of lignin in the production of biodegradable plastics, coatings, and composites, which could offer sustainable alternatives to conventional materials.

The valorization of lignin is one of the most promising and efficient pathways for lignin degradation into value-added components such as phenolics (vanillin being the best known) or hydrocarbon fuels after removing all oxygen components by hydrodeoxygenation (HDO), with consumption of a large amount of H₂. The potentials and promise of the big picture of the technology are huge, but the minute details need new technology. Keeping the large field of technology in mind, in an invention brief summary or discussion, the technology disclosed herein can be introduced by reviewing/discussing the following brief list of detailed features, which can be inter-combined with any other embodiment, example, detail, or aspect disclosed herein:

Feature 1: A method for selective partial depolymerization of natural lignin source, the method comprising the steps of: (1) obtaining a natural lignin source; (2) performing a photocatalytic activation of a β-O-4 bond in the natural lignin source by using a photocatalytic activation system comprising a catalyst comprising tetrabutylammonium decatungstate (TBADT) and a light source with light in an ultraviolet region shined on the natural lignin source; and (3) adding an exogenous electron mediator/scavenger system comprising an exogenous electron mediator to promote a cleavage or a bond-scission of the β-O-4 bond and/or an exogenous electron scavenger to promote an oxidation of the β-O-4 bond; whereby the addition of the exogenous electron mediator increases a yield of an oligomer but decreases an amount of carbonyl (C═O) groups in the oligomer and the addition of the exogenous electron scavenger increases the amount of carbonyl (C═O) groups in the oligomer and slightly changes the yield of the oligomer, wherein the yield of the oligomer is a weight of the oligomer divided by a weight of the natural lignin source.

Feature 2: The method of feature 1, wherein the natural lignin source is selected from a group consisting of hardwood, softwood, annual fibers, lignocellulosic biomass, wheat straw, rice straw, switchgrass, miscanthus, poplar wood, pine wood, corn stover, and bagasse.

Feature 3: The method of feature 1, wherein the photocatalytic activation system further comprises at least one of a solvent, a pH modifier, and a surfactant.

Feature 4: The method of feature 3, wherein the solvent is selected from a group consisting of water, acetonitrile, dimethylformamide, ethanol, acetone (AC), dichloromethane (DCM), dioxane, ethyl acetate (EA), hexane (Hex), methanol (MeOH), n-butylamine, and tetrahydrofuran (THF).

Feature 5: The method of feature 3, wherein the pH modifier is selected from a group consisting of an acid and a base, wherein the acid is selected from a group consisting of hydrochloric acid, sulfuric acid, phosphoric acid, and acetic acid, and wherein the base is selected from a group consisting of sodium hydroxide, potassium hydroxide, and ammonium hydroxide.

Feature 6: The method of feature 3, wherein the surfactant is selected from a group consisting of sodium dodecyl sulfate, cetyltrimethylammonium bromide, and Triton X-100.

Feature 7: The method of feature 1, wherein the light source emits light with a wavelength ranging from about 150 nm to about 800 nm, or about 200 nm to about 600 nm, or about 200 nm to about 400 nm.

Feature 8: The method of feature 1, wherein the exogenous electron mediator is selected from a group consisting of methyl viologen, benzyl viologen, 2,2′-bipyridine, and 9,10-diphenylanthracene (DPA).

Feature 9: The method of feature 1, wherein the exogenous electron scavenger is selected from a group consisting of oxygen, hydrogen peroxide, and ammonium persulfate.

Feature 10: The method of feature 1, wherein the oligomer has a number average molecular weight ranging from about 500 Da to about 10000 Da.

Feature 11: The method of feature 1, wherein the oligomer has a dispersity ranging from about 1.5 to about 3.5.

Feature 12: The method of feature 1, wherein the oligomer has a yield ranging from about 10 wt % to about 90 wt % based on the weight of the natural lignin source.

Feature 13: The method of feature 1, wherein the oligomer has an amount of carbonyl (C═O) groups ranging from about 0.1 mmol/g to about 5 mmol/g.

Feature 14: The method of feature 1, wherein the oligomer is further converted to a chemically recyclable polymer network.

Feature 15: The method of feature 14, wherein the chemically recyclable polymer network is selected from a group consisting of a polyurethane, a polyester, a polyamide, and a polycarbonate.

Feature 16: The method of feature 1, wherein the photocatalytic activation and the addition of the exogenous electron mediator/scavenger system are performed simultaneously or sequentially.

Feature 17: The method of feature 1, wherein the photocatalytic activation is performed at a temperature ranging from about 10° C. to about 150° C.

Feature 18: The method of feature 1, wherein the photocatalytic activation is performed for a duration ranging from about 1 hour to about 24 hours.

Feature 19: The method of feature 1, wherein the exogenous electron mediator/scavenger system is added in an amount ranging from about 0.1 mol % to about 10 mol % based on the moles of the β-O-4 bond in the natural lignin source.

Feature 20: The method of feature 1, wherein the exogenous electron mediator/scavenger system is added for a duration ranging from about 1 hour to about 24 hours.

Feature 21: The method of feature 1, wherein without added electron scavengers, TBADT is capable of extracting electrons and protons from the natural lignin; the TBADT acting as an oxidant, which will produce an amount of oxidation products (19.0%) even under a pure nitrogen atmosphere.

While the brief list of features above provides an introduction, it should be understood that any feature can be inter-combined with any detail, embodiment, aspect, or example disclosed herein. Other implementations are also described and recited herein. These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

Solely for the purpose of illustration, certain embodiments of the present invention are explained using examples in the drawings described below. It should be understood, however, that the invention is not limited to the precise arrangements, dimensions, and configurations shown. In the figures:

FIG. 1A shows the chemical structure of m6A (left) and hydrophobic binding pocket of YTHYTHDF1 (right).

FIG. 1B illustrates mechanisms of hydrogen-atom transfer to break the β-O-4 motif, which is abundant in native lignin.

FIG. 1C shows an overview of our strategy to first depolymerize native lignin and then repolymerize the resulting oligomers using dynamic covalent cross-linkers.

FIG. 1D shows an example of a novel method disclosed herein.

FIG. 2A illustrates the catalytic cycle of [W10O32]4- in the presence of an electron mediator, DPA, which leads to the scission of the β-O-4 motif.

FIG. 2B illustrates the catalytic cycle of [W10O32]4- in the presence of an electron scavenger, 02, which leads to the oxidation of the β-O-4 motif.

FIG. 3A shows two types of PPol reaction products in the presence of UV light: (1) oxidation and (2) bond scission.

FIG. 3B shows a comparison of the yields and distribution of different products with varying DPA amount.

FIG. 3C shows a comparison of the yields and distribution of different products with varying 02 concentration.

FIG. 4A shows a comparison of the yield and C═O stoichiometry obtained from photocatalytic degradation of native lignin with varying DPA amount.

FIG. 4B shows a comparison of the C═O stoichiometry obtained with varying atmosphere.

FIG. 4C shows a 2D-HSQC spectrum of organosolv lignin showing the integration of functional groups of interest.

FIG. 4D shows a 2D-HSQC spectrum of lignin oligomers showing the integration of functional groups of interest.

FIG. 5A shows a schematic illustration of the formation and recycling of lignin oligomer containing DPNs.

FIG. 5B and FIG. 5C show tensile tests comparing different triamine cross-linked networks.

FIG. 6 shows a gas chromatograph of the photocatalytic degradation of PPol.

FIG. 7 shows a comparison of the products of the photocatalysis of PPol using varying amounts of TBADT.

FIG. 8 shows a gas chromatograph of the photocatalytic degradation of PPol under 100% oxygen atmosphere.

FIG. 9 shows an SEC of catalysts, crude oligomers, and purified oligomers.

FIG. 10 shows an NMR of the hydrzaone formation for the quantification of carbonyl concentration in DMSO-d6 600 MHz.

FIG. 11 shows a comparison of the yields of the photocatalysis of native lignin to lignin oligomers under varying atmospheres.

FIG. 12 shows a full 2D-HSQC of organosolv lignin in DMSO-d6, 500 MHz.

FIG. 13 shows a full 2D-HSQC of lignin oligomers in DMSO-d6, 500 MHz.

FIG. 14 shows a quantification of remaining cellulose and hemicellulose after photocatalysis.

FIG. 15 shows a DMA analysis of original crosslinked TAEA network and recycled TAEA network.

FIG. 16A shows an example light setup used for photocatalysis.

FIG. 16B and FIG. 16C show example dimensions for a polytetrafluoroethylene (PTFE) mold used for creating tensile test and DMA samples.

FIG. 17A shows a table with a summary of PPol photocatalysis under varying conditions.

FIG. 17B shows a table with a summary of data.

FIG. 17C shows a table with a summary of native lignin photocatalysis results under varying conditions.

FIG. 18 shows a 1H NMR of copolymer filler before end group exchange in DMSO-d6, 600 MHz.

FIG. 19 shows a 1H NMR of copolymer filler after end group exchange in DMSO-d6, 600 MHz.

FIG. 20 shows an SEC trace of copolymer filler before end group exchange.

FIG. 21 shows an SEC trace of copolymer filler after end group exchange.

FIG. 22A and FIG. 22B (combined together) illustrate a process for optimizing lignin conversion to oligomers and polymers using photocatalytic activation and electron mediators/scavengers.

Any trademarks, images, likenesses, words, and depictions in the drawings and the disclosure are plainly in fair use and are provided solely for the purposes of illustration of the invention in view of an urgent need to treat subjects as further discussed in detail below. Any additional references can be included in entirety by mentioning herein.

### DETAILED DESCRIPTION OF THE INVENTION

The subject innovation is now described in some instances, when necessary, with reference to the drawings. In the following description, for purposes of explanation, numerous specific details are set forth in order to provide a thorough understanding of the present invention. It may be evident, however, that the present invention may be practiced without these specific details. In other instances, well-known structures, methods, and devices are shown in block diagram form or with illustrations in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail. In general, chemical terminology is found in the International Union of Pure and Applied Chemistry GoldBook. This disclosure is purposefully in commonly understood words, known to a person of skill in the art, but Merriam-Webster's Online Dictionary is used, when appropriate, for terms not specifically demonstrated herein or not known in the art.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation. The term “including” can be interchanged with “comprising”.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention. For example, a pharmaceutical formulation can consist essentially of an active agent and another active ingredient, meaning that a variety of excipients or other additives can be present in the formulation, but no other active pharmaceutical ingredient (API) is present in the formulation, except in formulations wherein an intended synergistic effect is demonstrated by the claims or examples herein (e.g., a formulation consisting essentially of one, two, or three ingredients or pharmaceutically acceptable salts thereof). In another example, a pharmaceutical formulation can consist essentially of an active agent and another ingredient, meaning that the formulation is provided in the form of a nasal spray, an inhaled formulation, an orally administered formulation, or an injection formulation, each of which is tailored for a fast-acting agent, therapeutic agent, or antidote but not tailored for long-term administration (e.g., as a simultaneous treatment). In another example, in the case of a preventative therapy to purposefully prevent a condition, the opposite, long-term combination therapy, can be referred to with “consisting essentially of”. In another example, the term “consisting essentially of” can also be exemplified by plain language provided in the claims.

The term “statistically significant” or “significantly” refers to statistical significance and generally means a two-standard deviation (2SD) or greater difference.

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed. and in Harrison, et al., Compendium of Synthetic Organic Methods, Vols. 1-8. Examples of representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Examples of representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a small molecule is less than 1500 MW or a large molecule not less than 1500 MW including, for example, biologics, oligonucleotides, peptides, systems of large molecules, oligosaccharides, and larger molecules. Any small molecule referred to herein can include atomic substitutions (and/or bonds) that are known in the art, for example, a radioactive isotope, a different element with similar characteristics, or a different bonding.

“Native lignin” refers to lignin in its natural state within plant cell walls, while “technical lignin” is the lignin extracted from wood pulp during paper production, which undergoes significant structural changes due to the harsh chemical treatments involved, making it less reactive and structurally altered compared to the native form; essentially, technical lignin is a byproduct of the pulping process, while native lignin is the lignin found naturally in plants.

In the embodiments discussed and in any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

As used herein, the word “method” can be made into a composition, a system, or a device, or vice versa. The technology contemplates vast compositions, system, devices, and scale up and is not limited by the methods or examples discussed herein. Other terms are defined herein within the description of the various aspects of the invention.

### Photocatalytic Valorization of Lignin

As an inedible component of biomass, lignin features rich functional groups that are desired for chemical syntheses. How to effectively depolymerize lignin without compromising the more valuable cellulose and hemicellulose has been a significant challenge. Existing biomass processing procedures either induce extensive condensation in lignin that greatly hinders its chemical utilization or focus on fully depolymerizing lignin to produce monomers that are difficult to separate for subsequent chemical synthesis.

Here, we report a new approach to selective partial depolymerization, which produces oligomers that can be readily converted to chemically recyclable polymer networks. The process takes advantage of the high selectivity of photocatalytic activation of the β-O-4 bond in lignin by tetrabutylammonium decatungstate (TBADT). The availability of exogenous electron mediators or scavengers promotes cleavage or oxidation of this bond, respectively, enabling high degrees of control over the depolymerization and the density of a key functional group, C═O, in the products. The resulting oligomers can then be readily utilized for the synthesis of polymer networks through reactions between C═O and branched —NH2 as a dynamic covalent cross-linker. Importantly, the resulting polymer network can be recycled to enable a circular economy of materials directly derived from biomass.

As an important component of lignocellulose, lignin offers numerous advantages as an attractive feedstock. For example, lignin is abundant, accounting for 15-40% of the total biomass; (1) it is rich in aromatic functionalities that are of great potential value for chemical synthesis and material fabrication; lignin is inedible, so its utilization will not compete with food needs. (2,3) However, existing biomass processing technologies prioritize cellulose and hemicellulose. (4) As a result, lignin has been significantly underutilized. (5) Consider the traditional pulping process as an example. The delignification methods produce the so-called technical lignin, which often leads to structural heterogeneity and undesired side reactions (e.g., condensation) and makes its subsequent chemical utilization challenging. (5,6)

Recently, an alternative lignin-first strategy has emerged to directly convert native lignin in lignocellulose into value-added chemicals. (4,7,8) For instance, reductive catalytic fractionation (RCF) as a lignin-first approach produces a mixture of low molecular weight compounds from native lignin. (9-13) However, the mixture produced by RCF is often difficult to separate. Moreover, RCF tends to destroy high-value functional groups such as carboxylic acids, aldehydes, and aromatic rings, undermining the value of these products as precursors for chemical syntheses. (9,14-16) Indeed, most RCF studies focus on retrieving the thermal energy of the products by using them as fuels.

Recognizing these challenges, researchers have recently turned their attention to depolymerizing native lignin under mild conditions. Successful examples have been demonstrated to utilize the hydrogen-atom transfer (HAT) reaction for selectively targeting the abundant β-1 and β-O-4 motifs. (17-25) A unique advantage offered by HAT is the ability to preserve the aromatics, ketones, and aldehydes. (17,21)

Nevertheless, earlier attempts of using HAT-based chemistries for lignin valorization have mostly focused on producing small molecules, which remain challenging to separate. (18,22) On the other hand, partial depolymerization of lignin has started to show its promise for the construction of functional materials, such as thermoset plastics, (26-29) elastomers, (30) or vitrimers. (31) Nevertheless, these initial materials are constructed from kraft lignin, (31) which has already undergone significant unwanted chemical modifications in the pulping process that affects its chemical integrity. (5,6)

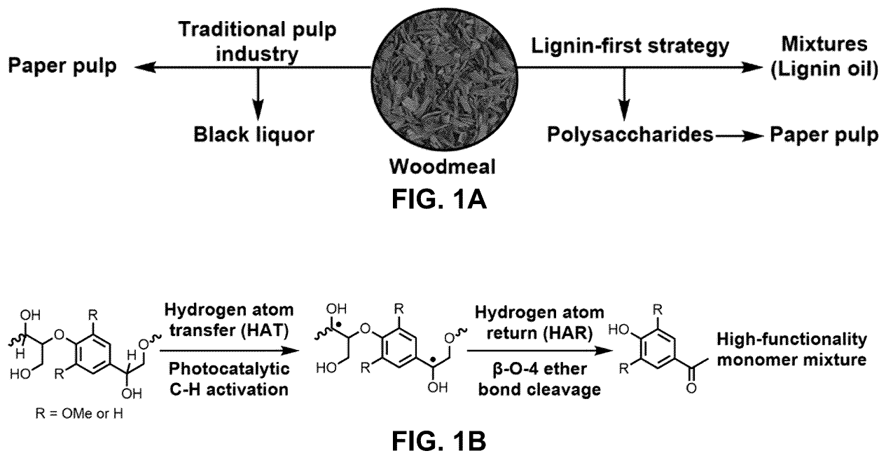

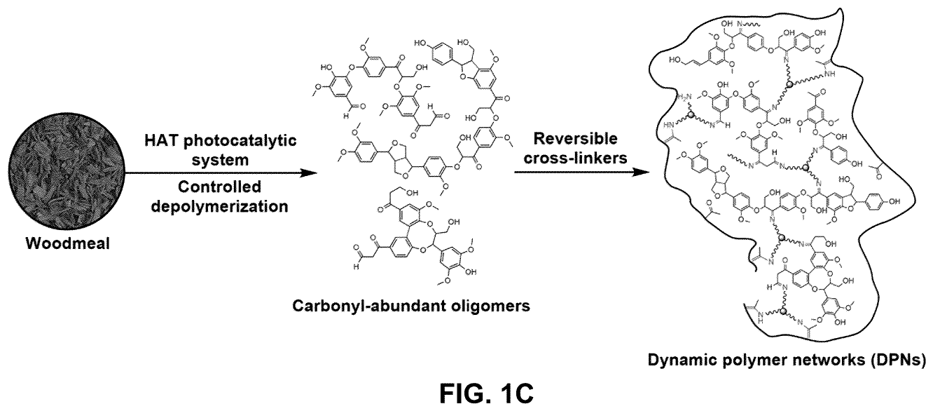

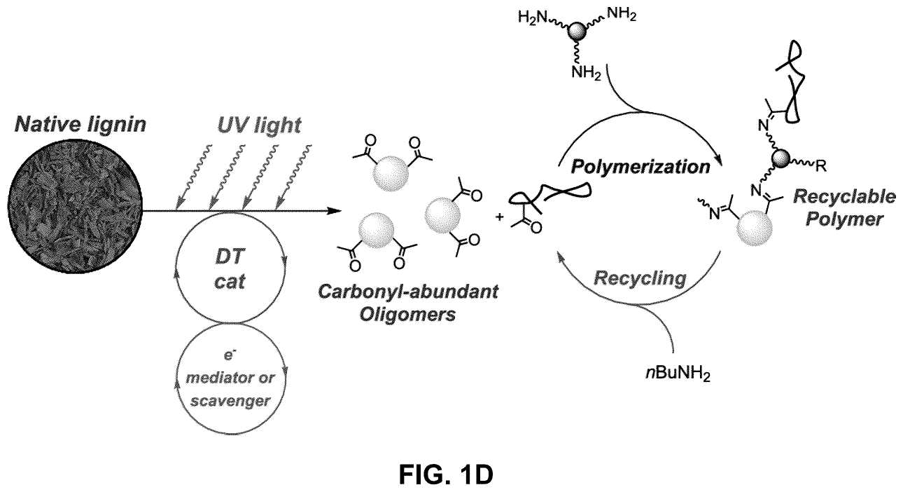

We see from this discussion the significance of a lignin-first approach, in which native lignin is selectively depolymerized for subsequent repolymerization. To preserve the high-value cellulose and hemicellulose, the depolymerization should take place under mild conditions. Here, we report a proof of this concept (FIG. 1A, FIG. 1B, FIG. 1C, FIG. 1D). Our work is inspired by previous reports on the cleavage of the β-O-4 motif in lignin via HAT. (18-23,25) To achieve the intricate balance between depolymerization and the introduction of functional groups necessary for the subsequent repolymerization, we employ an earth-abundant photocatalyst (namely, tetrabutylammonium decatungstate, or TBADT) (32) that enables HAT under mild conditions.

The reaction can be guided through either a bond-scission or an oxidation pathway through exogenous electron mediators or electron scavengers, respectively, thereby regulating the degree of depolymerization and introducing carbonyl functionality into the resulting oligomers. Repolymerization of the oligomers readily produced dynamic polymer networks (DPNs) that are capable of closed-loop chemical recycling toward a biomass-based circular plastic economy.

FIG. 1A, FIG. 1B, and FIG. 1C provide a schematic illustration of non-limiting examples of different strategies to process lignocellulose. FIG. 1A shows two industrial routes of processing woodmeal, namely, the pulp industry or the lignin oil industry. FIG. 1B illustrates mechanisms of hydrogen-atom transfer to break the β-O-4 motif, which is abundant in native lignin. FIG. 1C shows an overview of our strategy to first depolymerize native lignin and then repolymerize the resulting oligomers using dynamic covalent cross-linkers. FIG. 1D shows an example of a novel method disclosed herein.

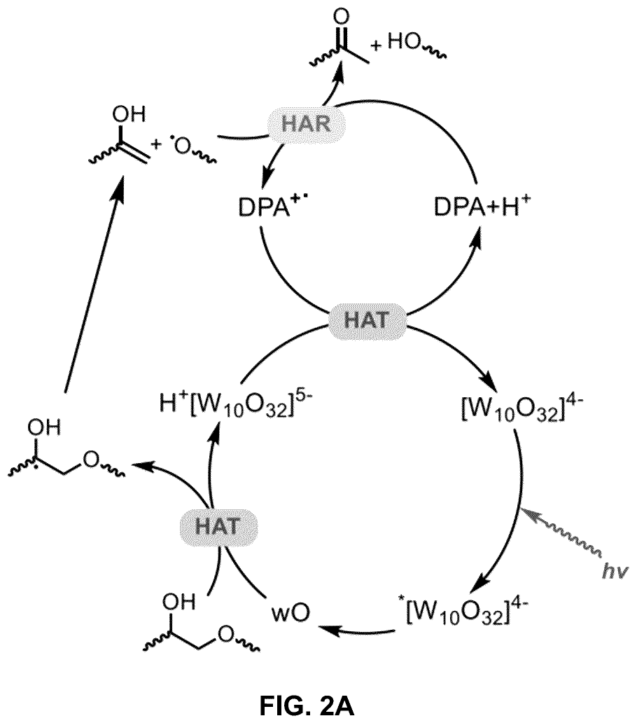

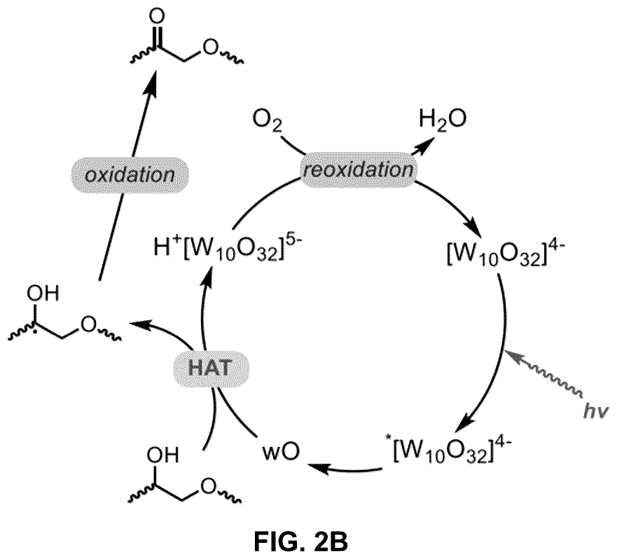

Results and Discussion: Inspired by recent reports that show decatungstate ([W₁₀O₃₂]⁴⁻), a polyoxometalate anion, as an effective hydrogen-atom abstraction (HAA) reagent, (33-37) we hypothesized that TBADT can be an efficient catalyst to directly target the abundant β-O-4 motifs in native lignin under mild conditions. Our proposed mechanism for the HAT mediated by TBADT involves hydrogen extraction from the substrate to result in an α-C radical upon irradiation, as shown in FIG. 2A and FIG. 2B. It can then lead to the scission of the β-C—O bond, producing an enol and an oxyl radical. The enol undergoes tautomerization to yield a ketone, and the oxyl radical receives the previously extracted hydrogen to produce an alcohol. (18-21) It is noted that bond scission could also take place between the α-C—C bond, yielding an aldehyde and a methyl ether. (17) In either case, the overall reaction involves a hydrogen-atom return (HAR). Previous reports have shown that the presence of an electron mediator, such as 9,10-diphenylanthracene (DPA), can lend its electron to the radical (38,39) and facilitate HAR to promote bond scission (FIG. 2A). In an alternative pathway, the α-C radical may be transformed into a stable carbonyl via oxidation, as shown in FIG. 2B, in which case an electron scavenger would be necessary to turn over the catalyst by extracting the hydrogen. (37)

FIG. 2A and FIG. 2B show two possible catalytic cycles of photocatalytic conversion of the β-O-4 motif with different cocatalysts. FIG. 2A illustrates the catalytic cycle of [W₁₀O₃₂]⁴⁻ in the presence of an electron mediator, DPA, which leads to the scission of the β-O-4 motif. FIG. 2B illustrates the catalytic cycle of [W₁₀O₃₂]⁴⁻ in the presence of an electron scavenger, 02, which leads to the oxidation of the β-O-4 motif.

To test the proposed mechanism involving the β-O-4 motif, we next carried out photocatalytic reactions on a model compound, 2-phenoxy-1-phenylethanol (PPol), which has been used as a testing platform for the study of lignin chemistry by other reports. (18,19,21,40,41) In a typical experiment, 50 μmol of PPol was mixed with 1.6 μmol of TBADT in 1 mL of acetonitrile, and the resulting solution was heated to 50° C. using a water bath under illuminated by a 200 W UV LED light centered at ca. 365 nm (see Examples below for more details). The reaction was performed under 1 bar of N₂.

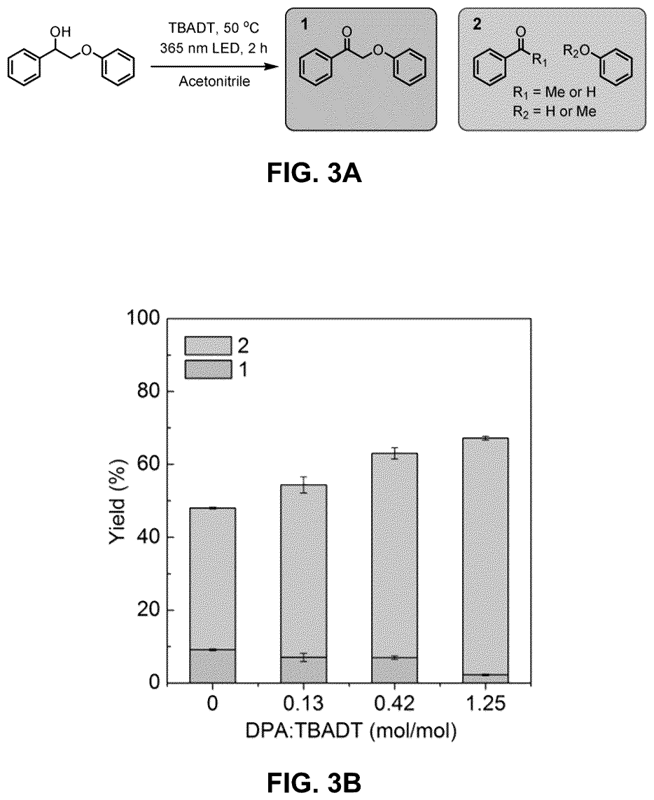

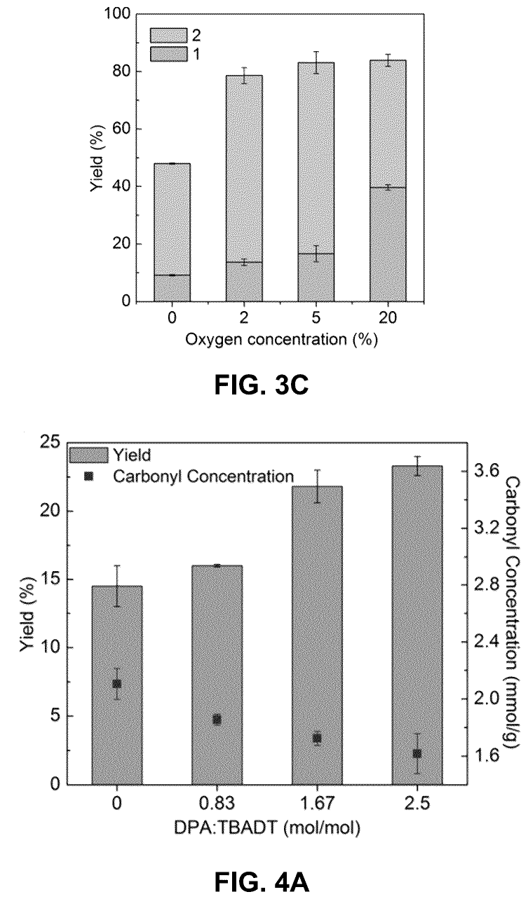

As discussed above, two types of reactions are expected from this system, a redox neutral process that breaks down the β-C—O (or the α-C—C) bond or an oxidation reaction that preserves the β-O-4 motif but produces a carbonyl (FIG. 3A). For a typical 2 h reaction, 4.56 μmol of 2-phenoxy-1-phenylethanone (PPone, compound 1 in FIG. 3A) was detected, accounting for 9.1% oxidation of the starting material (PPol). For the same reaction, 19.4 μmol of benzaldehyde or acetophenone (compounds 2 in FIG. 3A) or both was measured, reporting 38.9% bond scissions of the starting material. Also, 26.0 μmol of unreacted PPol was measured, which is consistent with the calculated conversion of 48.0% (FIG. 3B). Adding DPA as an electron mediator under otherwise identical conditions promoted the selectivity toward bond scission. Of the converted starting material, 81.0% underwent bond breaking when no DPA was added; the selectivity increased to 96.7% when 125 mol % of DPA (relative to TBADT) was used (FIG. 3B). Also increased was the total conversion, from 48.0% without DPA to 67.2% with 125 mol % of DPA. The increase in conversion was attributed to the improved TBADT turnover by DPA. As shown in FIG. 2A, when DPA⁺⁺ extracts hydrogen from the reduced TBADT, it facilitates the restoration of TBADT to the initial state, ready for the next catalytic cycle. Taken as a whole, it is concluded from this series of experiments that TBADT is effective in cleaving PPol and introducing carbonyl functional groups, and addition of electron mediators can further promote this reaction.

FIG. 3A, FIG. 3B, and FIG. 3C illustrate photocatalytic conversion of the model compound, PPol, under varying conditions. FIG. 3A shows two types of PPol reaction products in the presence of UV light: (1) oxidation and (2) bond scission. FIG. 3B shows a comparison of the yields and distribution of different products with varying DPA amount. FIG. 3C shows a comparison of the yields and distribution of different products with varying 02 concentration.

We propose that the carbonyl (C═O) groups can serve as a convenient reactive handle to form a dynamic imine bond by reacting with amines. (42-46) Therefore, it is of critical importance to control the density of C═O during the depolymerization of native lignin. Too few C═O groups will likely make it difficult to repolymerize the resulting oligomers; too many C═O groups would mean that the reaction pathway is shunted toward the oxidation pathway, causing incomplete depolymerization. A unique advantage offered by our photocatalytic depolymerization approach is the ability to control the reaction pathways by the introduction of exogenous electron scavengers.

When the reaction proceeds in a redox-neutral fashion, it favors the depolymerization of native lignin; when it undergoes oxidation, it is more effective in increasing C═O densities. Such a selectivity is new and significant because it will allow us to fine tune the degree of depolymerization of native lignin while controlling the density of key functional groups for subsequent repolymerization. To test this understanding, the following set of experiments (and continued in Examples below) was carried out on the model molecule of PPol. As shown in FIG. 3C, addition of 02 as an electron scavenger to the reaction system led to increased selectivity toward oxidation, changing the selectivity toward compound 1 from 19.0% with no 02 to 47.2% with 20% of 02 in N₂ under otherwise identical reaction conditions. The corresponding stoichiometry of C═O increased from 2.24 mmol/g without 02 to 3.94 mmol/g with 20% of 02. It was observed that even without intentionally added electron scavengers, TBADT was capable of extracting electrons and protons from the substrate, acting as an effective oxidant, which helped explain why an appreciable amount of oxidation products (19.0%) was present under a pure N₂ atmosphere.

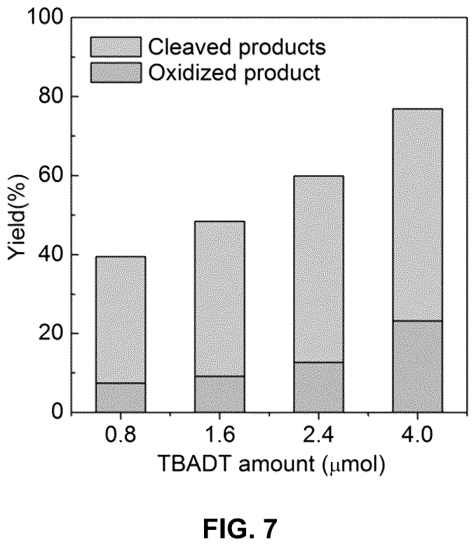

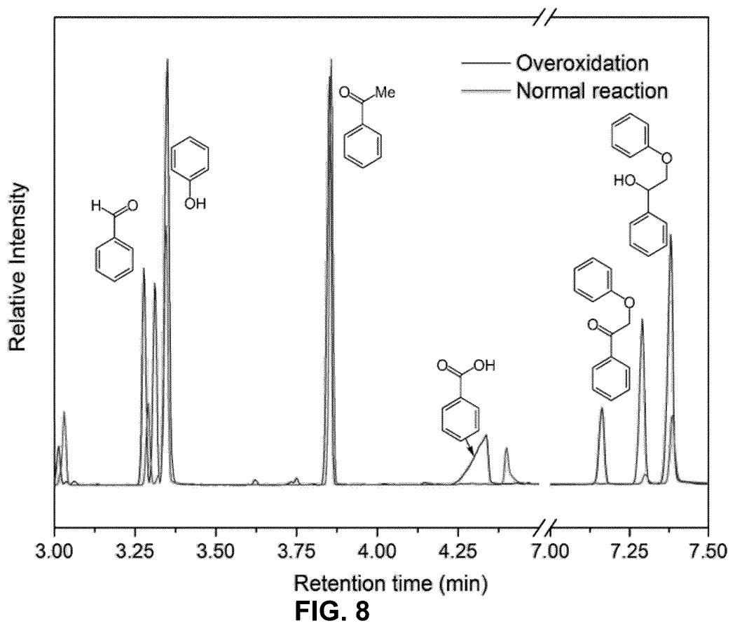

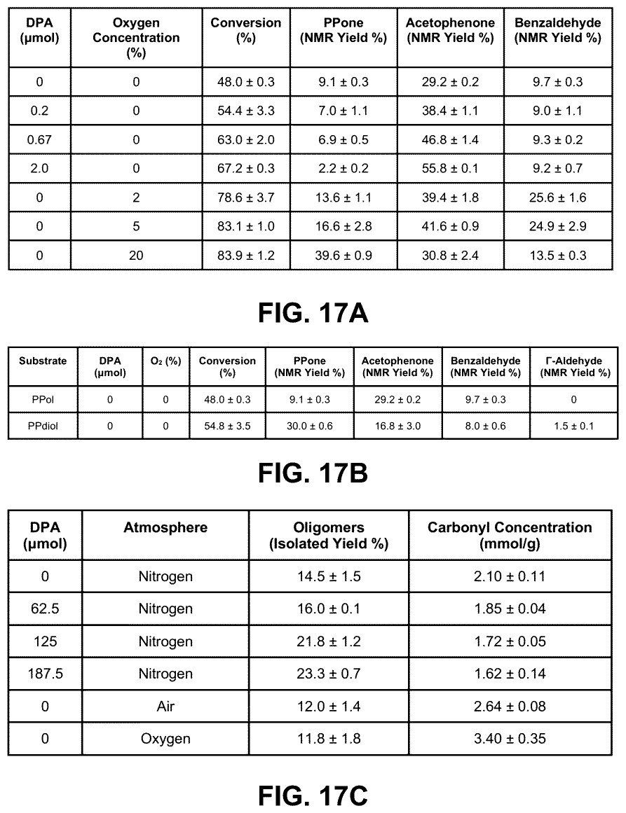

Our control experiments further proved that the concentration of oxidation products scaled with the amount of TBADT used (FIG. 7). Replacing all N₂ with 100% 02 led to overoxidation and diminished products of the desired compounds 1 or 2 (FIG. 8). It was also observed that the addition of 02 significantly improved the conversion of PPol. For instance, only 2% of 02 increased the conversion from 48.0% to 78.6%. In the absence of electron mediators, the improved conversion is likely due to more TBADT turnovers, as shown in FIG. 2B. These experiments carried out on the model compound, PPol, established that photocatalytic transformation of the β-O-4 motif can be achieved using TBADT following a HAT mechanism. It also showed that the reaction can be controlled between bond scission and oxidation. To further our understanding, we subjected another model system, 2-phenoxyl-1-phenylpropan-1,3-diol (PPdiol), to the same conditions and found no significant difference in conversion with similar product distributions (FIG. 17B Table). With this knowledge in hand, we next turned our attention to the valorization of native lignin (see Examples below).

In a detailed discussion of the technology herein, a list of embodiments in example details are provided below; any of the details provided below can be inter-changed with any example, feature, embodiment, aspect, or specification revealed herein at any other location:

Detail 1: A method for selective partial depolymerization of natural lignin source, the method comprising the steps of: (1) obtaining a natural lignin source; (2) performing a photocatalytic activation of a β-O-4 bond in the natural lignin source by using a photocatalytic activation system comprising a catalyst comprising tetrabutylammonium decatungstate (TBADT) and a light source with light in an ultraviolet region shined on the natural lignin source; and (3) adding an exogenous electron mediator/scavenger system comprising an exogenous electron mediator to promote a cleavage or a bond-scission of the β-O-4 bond and/or an exogenous electron scavenger to promote an oxidation of the β-O-4 bond; whereby the addition of the exogenous electron mediator increases a yield of an oligomer but decreases an amount of carbonyl (C═O) groups in the oligomer and the addition of the exogenous electron scavenger increases the amount of carbonyl (C═O) groups in the oligomer and slightly changes the yield of the oligomer, wherein the yield of the oligomer is a weight of the oligomer divided by a weight of the natural lignin source.

Detail 2: The method of detail 1, wherein the natural lignin source is selected from a group consisting of hardwood, softwood, annual fibers, lignocellulosic biomass, wheat straw, rice straw, switchgrass, miscanthus, poplar wood, pine wood, corn stover, and bagasse.

Detail 3: The method of detail 1, wherein the photocatalytic activation system further comprises at least one of a solvent, a pH modifier, and a surfactant.

Detail 4: The method of detail 3, wherein the solvent is selected from a group consisting of water, acetonitrile, dimethylformamide, ethanol, acetone (AC), dichloromethane (DCM), dioxane, ethyl acetate (EA), hexane (Hex), methanol (MeOH), n-butylamine, and tetrahydrofuran (THF).

Detail 5: The method of detail 3, wherein the pH modifier is selected from a group consisting of an acid and a base, wherein the acid is selected from a group consisting of hydrochloric acid, sulfuric acid, phosphoric acid, and acetic acid, and wherein the base is selected from a group consisting of sodium hydroxide, potassium hydroxide, and ammonium hydroxide.

Detail 6: The method of detail 3, wherein the surfactant is selected from a group consisting of sodium dodecyl sulfate, cetyltrimethylammonium bromide, and Triton X-100.

Detail 7: The method of detail 1, wherein the light source emits light with a wavelength ranging from about 150 nm to about 800 nm, optionally about 200 nm to about 600 nm, or about 200 nm to about 400 nm.

Detail 8: The method of detail 1, wherein the exogenous electron mediator is selected from a group consisting of methyl viologen, benzyl viologen, 2,2′-bipyridine, and 9,10-diphenylanthracene (DPA).

Detail 9: The method of detail 1, wherein the exogenous electron scavenger is selected from a group consisting of oxygen, hydrogen peroxide, and ammonium persulfate.

Detail 10: The method of detail 1, wherein the oligomer has a number average molecular weight ranging from about 500 Da to about 10000 Da.

Detail 11: The method of detail 1, wherein the oligomer has a dispersity ranging from about 1.5 to about 3.5.

Detail 12: The method of detail 1, wherein the oligomer has a yield ranging from about 10 wt % to about 90 wt % based on the weight of the natural lignin source.

Detail 13: The method of detail 1, wherein the oligomer has an amount of carbonyl (C═O) groups ranging from about 0.1 mmol/g to about 5 mmol/g.

Detail 14: The method of detail 1, wherein the oligomer is further converted to a chemically recyclable polymer network.

Detail 15: The method of detail 14, wherein the chemically recyclable polymer network is selected from a group consisting of a polyurethane, a polyester, a polyamide, and a polycarbonate.

Detail 16: The method of detail 1, wherein the photocatalytic activation and the addition of the exogenous electron mediator/scavenger system are performed simultaneously or sequentially.

Detail 17: The method of detail 1, wherein the photocatalytic activation is performed at a temperature ranging from about 10° C. to about 150° C.

Detail 18: The method of detail 1, wherein the photocatalytic activation is performed for a duration ranging from about 1 hour to about 24 hours.

Detail 19: The method of detail 1, wherein the exogenous electron mediator/scavenger system is added in an amount ranging from about 0.1 mol % to about 10 mol % based on the moles of the β-O-4 bond in the natural lignin source.

Detail 20: The method of detail 1, wherein without added electron scavengers, TBADT is capable of extracting electrons and protons from the natural lignin; the TBADT acting as an oxidant, which will produce an amount of oxidation products (19.0%) even under a pure nitrogen atmosphere.

Detail 21: A method for selective partial depolymerization of natural lignin source, the method comprising: obtaining a natural lignin source; performing a photocatalytic activation of a β-O-4 bond in the natural lignin source by using a photocatalytic activation system comprising a catalyst comprising tetrabutylammonium decatungstate (TBADT) and a light source with light in an ultraviolet region shined on the natural lignin source; and adding an exogenous electron mediator/scavenger system comprising an exogenous electron mediator to promote a cleavage or a bond-scission of the β-O-4 bond and/or an exogenous electron scavenger to promote an oxidation of the β-O-4 bond; whereby the addition of the exogenous electron mediator increases a yield of an oligomer but decreases an amount of carbonyl (C═O) groups in the oligomer and the addition of the exogenous electron scavenger increases the amount of carbonyl (C═O) groups in the oligomer and slightly changes the yield of the oligomer, wherein the yield of the oligomer is a weight of the oligomer divided by a weight of the natural lignin source.

Detail 22: The method of detail 21, wherein the natural lignin source is selected from a group consisting of hardwood comprising oak, maple, birch, poplar, eucalyptus, and combinations thereof; softwood comprising pine, spruce, fir, cedar, redwood, and combinations thereof; annual fibers comprising wheat straw, rice straw, corn stover, sugarcane bagasse, bamboo, kenaf, jute, sisal, flax, hemp, ramie, abaca, and combinations thereof; and lignocellulosic biomass comprising agricultural residues, forestry residues, energy crops, municipal solid waste, and combinations thereof, wherein the natural lignin source has a lignin content ranging from about 10 wt % to about 50 wt % based on the total weight of the natural lignin source, and wherein the natural lignin source is in a form of chips, shavings, sawdust, powder, or a combination thereof with a particle size ranging from about 0.1 mm to about 10 mm.

Detail 23: The method of detail 21, wherein the photocatalytic activation system further comprises at least one of a solvent selected from a group consisting of water, acetonitrile, dimethylformamide, dimethyl sulfoxide, ethanol, methanol, isopropanol, tetrahydrofuran, dichloromethane, chloroform, ethyl acetate, acetone, and combinations thereof in an amount ranging from about 50 wt % to about 99 wt % based on the total weight of the photocatalytic activation system; a pH modifier selected from a group consisting of an acid comprising hydrochloric acid, sulfuric acid, nitric acid, phosphoric acid, acetic acid, formic acid, and combinations thereof and a base comprising sodium hydroxide, potassium hydroxide, ammonium hydroxide, sodium carbonate, potassium carbonate, and combinations thereof in an amount ranging from about 0.01 wt % to about 10 wt % based on the total weight of the photocatalytic activation system to adjust the pH of the photocatalytic activation system to a range from about 2 to about 12; and a surfactant selected from a group consisting of sodium dodecyl sulfate, cetyltrimethylammonium bromide, Triton X-100, Tween 20, Tween 80, Pluronic F-127, Brij 35, Brij 58, Brij 98, Igepal CO-630, Igepal CO-890, and combinations thereof in an amount ranging from about 0.01 wt % to about 10 wt % based on the total weight of the photocatalytic activation system.

Detail 24: The method of detail 23, wherein the solvent is selected from a group consisting of water, acetonitrile, dimethylformamide, and combinations thereof in an amount ranging from about 70 wt % to about 95 wt % based on the total weight of the photocatalytic activation system.

Detail 25: The method of detail 23, wherein the pH modifier is selected from a group consisting of hydrochloric acid, sulfuric acid, sodium hydroxide, potassium hydroxide, and combinations thereof in an amount ranging from about 0.1 wt % to about 5 wt % based on the total weight of the photocatalytic activation system to adjust the pH of the photocatalytic activation system to a range from about 3 to about 10.

Detail 26: The method of detail 23, wherein the surfactant is selected from a group consisting of sodium dodecyl sulfate, cetyltrimethylammonium bromide, Triton X-100, and combinations thereof in an amount ranging from about 0.1 wt % to about 5 wt % based on the total weight of the photocatalytic activation system.

Detail 27: The method of detail 21, wherein the light source emits light with a wavelength ranging from about 250 nm to about 380 nm at an intensity ranging from about 10 mW/cm2 to about 1000 mW/cm2, and wherein the light source is selected from a group consisting of a low-pressure mercury lamp, a medium-pressure mercury lamp, a high-pressure mercury lamp, a xenon lamp, a metal halide lamp, a light-emitting diode (LED), and combinations thereof.

Detail 28: The method of detail 21, wherein the exogenous electron mediator is selected from a group consisting of methyl viologen, benzyl viologen, 2,2′-bipyridine, 9,10-diphenylanthracene (DPA), and combinations thereof in an amount ranging from about 0.01 mol % to about 10 mol % based on the moles of the β-O-4 bond in the natural lignin source, and wherein the exogenous electron mediator has a reduction potential ranging from about −0.5 V to about −1.5 V vs. a standard hydrogen electrode (SHE).

Detail 29: The method of detail 21, wherein the exogenous electron scavenger is selected from a group consisting of oxygen, hydrogen peroxide, ammonium persulfate, and combinations thereof in an amount ranging from about 0.01 mol % to about 10 mol % based on the moles of the β-O-4 bond in the natural lignin source, and wherein the exogenous electron scavenger has an oxidation potential ranging from about +0.5 V to about +2.0 V vs. a standard hydrogen electrode (SHE).

Detail 30: The method of detail 21, wherein the oligomer has a number average molecular weight ranging from about 1000 Da to about 5000 Da as determined by gel permeation chromatography (GPC) using polystyrene standards.

Detail 31: The method of detail 21, wherein the oligomer has a dispersity ranging from about 2.0 to about 3.0 as determined by gel permeation chromatography (GPC) using polystyrene standards.

Detail 32: The method of detail 21, wherein the oligomer has a yield ranging from about 30 wt % to about 70 wt % based on the weight of the natural lignin source as determined by gravimetric analysis.

Detail 33: The method of detail 21, wherein the oligomer has an amount of carbonyl (C═O) groups ranging from about 0.5 mmol/g to about 3 mmol/g as determined by 31P NMR spectroscopy using 2-chloro-4,4,5,5-tetramethyl-1,3,2-dioxaphospholane as a phosphorylating agent.

Detail 34: The method of detail 21, wherein the oligomer is further converted to a chemically recyclable polymer network by reacting the oligomer with at least one of a diisocyanate, a diacid chloride, a dianhydride, a diepoxide, a diamine, a diol, and combinations thereof in the presence of a catalyst and a solvent at a temperature ranging from about 20° C. to about 200° C. for a duration ranging from about 1 hour to about 48 hours.

Detail 35: The method of detail 34, wherein the chemically recyclable polymer network is selected from a group consisting of a polyurethane formed by reacting the oligomer with a diisocyanate selected from a group consisting of hexamethylene diisocyanate, isophorone diisocyanate, methylene diphenyl diisocyanate, toluene diisocyanate, and combinations thereof; a polyester formed by reacting the oligomer with a diacid chloride selected from a group consisting of sebacoyl chloride, terephthaloyl chloride, isophthaloyl chloride, phthaloyl chloride, and combinations thereof or a dianhydride selected from a group consisting of pyromellitic dianhydride, 3,3′,4,4′-benzophenonetetracarboxylic dianhydride, 4,4′-oxydiphthalic anhydride, and combinations thereof; a polyamide formed by reacting the oligomer with a diamine selected from a group consisting of hexamethylenediamine, ethylenediamine, phenylenediamine, xylylenediamine, and combinations thereof; and a polycarbonate formed by reacting the oligomer with a diol selected from a group consisting of bisphenol A, bisphenol F, bisphenol S, 1,4-cyclohexanedimethanol, and combinations thereof and a diepoxide selected from a group consisting of bisphenol A diglycidyl ether, bisphenol F diglycidyl ether, 1,4-butanediol diglycidyl ether, and combinations thereof.

Detail 36: The method of detail 21, wherein the photocatalytic activation and the addition of the exogenous electron mediator/scavenger system are performed simultaneously in a one-pot reaction or sequentially in a two-step reaction, and wherein the one-pot reaction or the two-step reaction is carried out in a batch reactor, a continuous flow reactor, or a combination thereof.

Detail 37: The method of detail 21, wherein the photocatalytic activation is performed at a temperature ranging from about 20° C. to about 60° C. and a pressure ranging from about 1 atm to about 10 atm.

Detail 38: The method of detail 21, wherein the photocatalytic activation is performed for a duration ranging from about 2 hours to about 12 hours with a stirring speed ranging from about 100 rpm to about 1000 rpm.

Detail 39: The method of detail 21, wherein the exogenous electron mediator/scavenger system is added in an amount ranging from about 0.5 mol % to about 5 mol % based on the moles of the β-O-4 bond in the natural lignin source, and wherein the exogenous electron mediator/scavenger system is added in a solid form, a liquid form, a gaseous form, or a combination thereof.

Detail 40: The method of detail 21, wherein the exogenous electron mediator/scavenger system is added for a duration ranging from about 2 hours to about 12 hours with a stirring speed ranging from about 100 rpm to about 1000 rpm, and wherein the exogenous electron mediator/scavenger system is added in one portion, in multiple portions, or continuously during the photocatalytic activation.

Detail 41: A method for selective partial depolymerization of natural lignin source, the method comprising the steps of: (1) obtaining a natural lignin source, wherein the natural lignin source is derived from a plant biomass material selected from the group consisting of wood, agricultural residues, energy crops, and combinations thereof, and wherein the natural lignin source has a lignin content ranging from about 10 wt % to about 50 wt % based on the total weight of the plant biomass material; (2) performing a photocatalytic activation of a β-O-4 bond in the natural lignin source by using a photocatalytic activation system comprising a catalyst comprising tetrabutylammonium decatungstate (TBADT) in an amount ranging from about 0.1 mol % to about 10 mol % based on the moles of the β-O-4 bond in the natural lignin source and a light source with light in an ultraviolet region having a wavelength ranging from about 200 nm to about 400 nm shined on the natural lignin source for a duration ranging from about 1 hour to about 24 hours at a temperature ranging from about 10° C. to about 150° C.; and (3) adding an exogenous electron mediator/scavenger system comprising an exogenous electron mediator selected from the group consisting of methyl viologen, benzyl viologen, 2,2′-bipyridine, 9,10-diphenylanthracene (DPA), and combinations thereof to promote a cleavage or a bond-scission of the β-O-4 bond and/or an exogenous electron scavenger selected from the group consisting of oxygen, hydrogen peroxide, ammonium persulfate, and combinations thereof to promote an oxidation of the β-O-4 bond, wherein the exogenous electron mediator/scavenger system is added in an amount ranging from about 0.1 mol % to about 10 mol % based on the moles of the β-0-4 bond in the natural lignin source for a duration ranging from about 1 hour to about 24 hours; whereby the addition of the exogenous electron mediator increases a yield of an oligomer ranging from about 10 wt % to about 90 wt % based on the weight of the natural lignin source but decreases an amount of carbonyl (C═O) groups in the oligomer ranging from about 0.1 mmol/g to about 5 mmol/g and the addition of the exogenous electron scavenger increases the amount of carbonyl (C═O) groups in the oligomer ranging from about 0.1 mmol/g to about 5 mmol/g and slightly changes the yield of the oligomer ranging from about 10 wt % to about 90 wt % based on the weight of the natural lignin source, wherein the yield of the oligomer is a weight of the oligomer divided by a weight of the natural lignin source.

Detail 42: The method of detail 41, wherein the natural lignin source is selected from a group consisting of hardwood, softwood, annual fibers, lignocellulosic biomass, wheat straw, rice straw, switchgrass, miscanthus, poplar wood, pine wood, corn stover, bagasse, and combinations thereof, and wherein the natural lignin source has a moisture content ranging from about 1 wt % to about 20 wt % based on the total weight of the natural lignin source and an average particle size ranging from about 0.01 mm to about 10 mm.

Detail 43: The method of detail 41, wherein the photocatalytic activation system further comprises at least one of a solvent selected from the group consisting of water, acetonitrile, dimethylformamide, ethanol, acetone (AC), dichloromethane (DCM), dioxane, ethyl acetate (EA), hexane (Hex), methanol (MeOH), n-butylamine, tetrahydrofuran (THF), and combinations thereof in an amount ranging from about 10 wt % to about 99 wt % based on the total weight of the photocatalytic activation system, a pH modifier selected from the group consisting of an acid selected from the group consisting of hydrochloric acid, sulfuric acid, phosphoric acid, acetic acid, and combinations thereof and a base selected from the group consisting of sodium hydroxide, potassium hydroxide, ammonium hydroxide, and combinations thereof in an amount sufficient to adjust the pH of the photocatalytic activation system to a range of about 2 to about 12, and a surfactant selected from the group consisting of sodium dodecyl sulfate, cetyltrimethylammonium bromide, Triton X-100, and combinations thereof in an amount ranging from about 0.1 wt % to about 10 wt % based on the total weight of the photocatalytic activation system.

Detail 44: The method of detail 43, wherein the solvent is selected from a group consisting of water, acetonitrile, dimethylformamide, ethanol, acetone (AC), dichloromethane (DCM), dioxane, ethyl acetate (EA), hexane (Hex), methanol (MeOH), n-butylamine, tetrahydrofuran (THF), and combinations thereof, and wherein the solvent has a boiling point ranging from about 50° C. to about 200° C. and a polarity index ranging from about 1 to about 10.

Detail 45: The method of detail 43, wherein the pH modifier is selected from a group consisting of an acid selected from the group consisting of hydrochloric acid, sulfuric acid, phosphoric acid, acetic acid, and combinations thereof and a base selected from the group consisting of sodium hydroxide, potassium hydroxide, ammonium hydroxide, and combinations thereof, and wherein the pH modifier is added in an amount ranging from about 0.1 mol % to about 20 mol % based on the moles of the natural lignin source.

Detail 46: The method of detail 43, wherein the surfactant is selected from a group consisting of sodium dodecyl sulfate, cetyltrimethylammonium bromide, Triton X-100, and combinations thereof, and wherein the surfactant has a hydrophilic-lipophilic balance (HLB) value ranging from about 5 to about 20 and a critical micelle concentration (CMC) ranging from about 0.1 mM to about 10 mM.

Detail 47: The method of detail 41, wherein the light source emits light with a wavelength ranging from about 200 nm to about 400 nm, and wherein the light source has a power ranging from about 1 mW to about 1000 mW and an intensity ranging from about 1 mW/cm2 to about 100 mW/cm2.

Detail 48: The method of detail 41, wherein the exogenous electron mediator is selected from a group consisting of methyl viologen, benzyl viologen, 2,2′-bipyridine, 9,10-diphenylanthracene (DPA), and combinations thereof, and wherein the exogenous electron mediator has a reduction potential ranging from about −1.0 V to about 0.5 V vs. standard hydrogen electrode (SHE).

Detail 49: The method of detail 41, wherein the exogenous electron scavenger is selected from a group consisting of oxygen, hydrogen peroxide, ammonium persulfate, and combinations thereof, and wherein the exogenous electron scavenger has an oxidation potential ranging from about 0.5 V to about 2.0 V vs. standard hydrogen electrode (SHE).

Detail 50: The method of detail 41, wherein the oligomer has a number average molecular weight ranging from about 500 Da to about 10000 Da, and wherein the oligomer has a weight average molecular weight ranging from about 1000 Da to about 50000 Da.

Detail 51: The method of detail 41, wherein the oligomer has a dispersity ranging from about 1.5 to about 3.5, and wherein the dispersity is a ratio of the weight average molecular weight to the number average molecular weight of the oligomer.

Detail 52: The method of detail 41, wherein the oligomer has a yield ranging from about 10 wt % to about 90 wt % based on the weight of the natural lignin source, and wherein the oligomer has a purity ranging from about 50 wt % to about 99 wt % based on the total weight of the oligomer.

Detail 53: The method of detail 41, wherein the oligomer has an amount of carbonyl (C═O) groups ranging from about 0.1 mmol/g to about 5 mmol/g, and wherein the amount of carbonyl (C═O) groups is determined by a quantitative 13C NMR analysis or a titration method using hydroxylamine hydrochloride.

Detail 54: The method of detail 41, wherein the oligomer is further converted to a chemically recyclable polymer network, and wherein the chemically recyclable polymer network has a crosslink density ranging from about 0.1 mol % to about 10 mol % based on the moles of the monomers used to form the chemically recyclable polymer network.

Detail 55: The method of detail 54, wherein the chemically recyclable polymer network is selected from a group consisting of a polyurethane, a polyester, a polyamide, a polycarbonate, and combinations thereof, and wherein the chemically recyclable polymer network has a glass transition temperature (Tg) ranging from about −50° C. to about 200° C. and a decomposition temperature (Td) ranging from about 200° C. to about 500° C.

Detail 56: The method of detail 41, wherein the photocatalytic activation and the addition of the exogenous electron mediator/scavenger system are performed simultaneously or sequentially, and wherein the photocatalytic activation and the addition of the exogenous electron mediator/scavenger system are performed in a batch reactor, a continuous flow reactor, or a semi-batch reactor.

Detail 57: The method of detail 41, wherein the photocatalytic activation is performed at a temperature ranging from about 10° C. to about 150° C., and wherein the photocatalytic activation is performed at a pressure ranging from about 0.1 atm to about 10 atm.

Detail 58: The method of detail 41, wherein the photocatalytic activation is performed for a duration ranging from about 1 hour to about 24 hours, and wherein the photocatalytic activation is performed under an inert atmosphere selected from the group consisting of nitrogen, argon, helium, and combinations thereof or under an air atmosphere.

Detail 59: The method of detail 41, wherein the exogenous electron mediator/scavenger system is added in an amount ranging from about 0.1 mol % to about 10 mol % based on the moles of the β-O-4 bond in the natural lignin source, and wherein the exogenous electron mediator/scavenger system is added as a solid, a liquid, or a gas.

Detail 60: The method of detail 41, wherein without added electron scavengers, TBADT is capable of extracting electrons and protons from the natural lignin; the TBADT acting as an oxidant, which will produce an amount of oxidation products (19.0%) even under a pure nitrogen atmosphere, and wherein the oxidation products comprise vanillin, syringaldehyde, acetovanillone, acetosyringone, and combinations thereof.

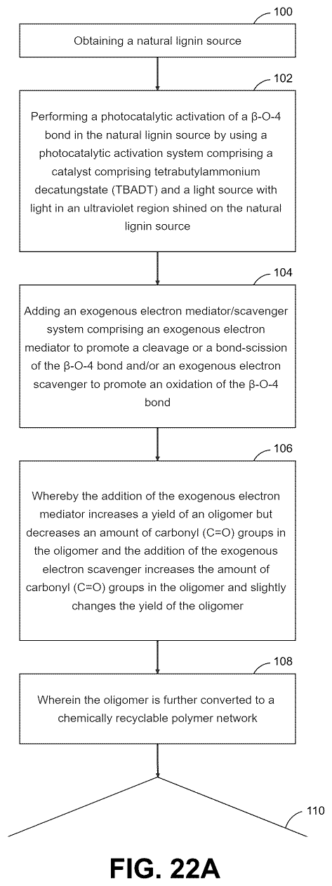

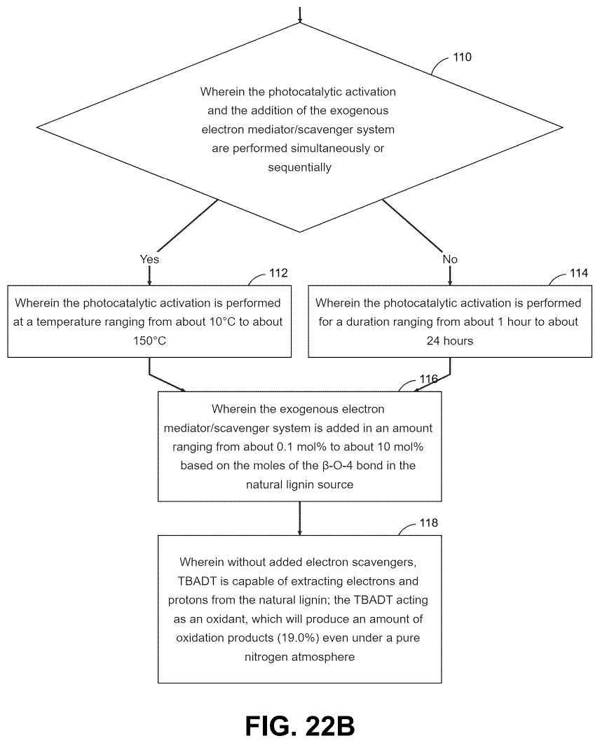

While keeping in mind the details, features, aspects, embodiments, and examples above (and the data Examples below), FIG. 22A and FIG. 22B provide a flowchart illustrating a method in step 100 for obtaining a natural lignin source, according to an embodiment. At step 100, a natural lignin source may be obtained, which can be selected from a variety of materials such as hardwood, softwood, annual fibers, lignocellulosic biomass, wheat straw, rice straw, switchgrass, miscanthus, poplar wood, pine wood, corn stover, and bagasse. This step may serve to provide the raw material for the subsequent depolymerization process. The selection of the natural lignin source may influence the efficiency and outcome of the depolymerization, as different sources may have varying compositions and structures. The process of obtaining the natural lignin source may involve sourcing, collecting, and preparing the material to ensure it is suitable for the photocatalytic activation that follows. The natural lignin source may be prepared in a manner that optimizes its exposure to the photocatalytic activation system, which may include considerations of particle size, moisture content, and purity. This preparation may ensure that the lignin is in a form that allows for effective interaction with the catalyst and light source used in the subsequent steps. The method may also consider the environmental and economic aspects of sourcing the natural lignin, aiming to utilize sustainable and cost-effective materials. The obtained natural lignin source may then be ready for the next step in the process, which involves photocatalytic activation to facilitate the selective partial depolymerization of the lignin.

In the context of step 102, the process may involve performing a photocatalytic activation of the β-O-4 bond within the natural lignin source. This activation may be facilitated by a photocatalytic activation system, which may include a catalyst such as tetrabutylammonium decatungstate (TBADT) and a light source emitting ultraviolet light. The ultraviolet light may be shined on the natural lignin source to provide the energy for the activation process. The photocatalytic activation system may further comprise additional components such as a solvent, a pH modifier, and a surfactant, which may enhance the activation process. The solvent may be selected from a variety of options, including water, acetonitrile, and dimethylformamide, among others, to suit the specific requirements of the reaction. The pH modifier may be an acid or a base, such as hydrochloric acid or sodium hydroxide, to adjust the pH to optimal levels for the reaction. The surfactant may be chosen from compounds like sodium dodecyl sulfate or Triton X-100 to improve the interaction between the catalyst and the lignin source. The light source may emit light with a wavelength ranging from about 200 nm to about 400 nm, which may be effective for the activation of the β-O-4 bond. This step may set the stage for subsequent reactions that may involve the addition of exogenous electron mediators or scavengers to further manipulate the chemical structure of the lignin, ultimately leading to the production of oligomers with desired properties.

In step 104, the process may involve the addition of an exogenous electron mediator/scavenger system to the natural lignin source. This system may include an exogenous electron mediator, which can promote the cleavage or bond-scission of the β-O-4 bond, and/or an exogenous electron scavenger, which may facilitate the oxidation of the β-O-4 bond. The exogenous electron mediator may be selected from compounds such as methyl viologen, benzyl viologen, 2,2′-bipyridine, and 9,10-diphenylanthracene (DPA). These mediators may enhance the yield of the oligomer by promoting the cleavage of the β-O-4 bond, potentially leading to a decrease in the amount of carbonyl (C═O) groups in the oligomer. Conversely, the exogenous electron scavenger, which may be chosen from oxygen, hydrogen peroxide, and ammonium persulfate, can increase the amount of carbonyl (C═O) groups in the oligomer while only slightly altering the yield. The addition of these components may be for achieving the balance between oligomer yield and carbonyl group content, which is for the subsequent conversion of the oligomer into chemically recyclable polymer networks. The process may be designed to allow for the simultaneous or sequential addition of the exogenous electron mediator and scavenger, providing flexibility in the method for selective partial depolymerization of natural lignin.

In the context of step 106, the process may involve the addition of an exogenous electron mediator and an exogenous electron scavenger to influence the characteristics of the resulting oligomer. The exogenous electron mediator may be added to increase the yield of the oligomer, which is defined as the weight of the oligomer divided by the weight of the natural lignin source. This addition may also result in a decrease in the amount of carbonyl (C═O) groups present in the oligomer. Conversely, the addition of an exogenous electron scavenger may lead to an increase in the amount of carbonyl (C═O) groups within the oligomer, while only slightly altering the yield of the oligomer. The oligomer produced through this method may possess a number average molecular weight ranging from about 500 Da to about 10000 Da, a dispersity ranging from about 1.5 to about 3.5, a yield ranging from about 10 wt % to about 90 wt % based on the weight of the natural lignin source, and an amount of carbonyl (C═O) groups ranging from about 0.1 mmol/g to about 5 mmol/g. The method for selective partial depolymerization of natural lignin may facilitate the production of an oligomer that can be readily converted to chemically recyclable polymer networks. The process may be designed to optimize the balance between oligomer yield and the presence of carbonyl groups, depending on the desired application of the oligomer.

In step 108, the oligomer may be further converted into a chemically recyclable polymer network. This conversion process may involve the transformation of the oligomer into a network that can potentially be broken down and reused, thus contributing to sustainability. The chemically recyclable polymer network may be selected from a group consisting of polyurethane, polyester, polyamide, and polycarbonate. These materials may offer various properties and applications, depending on the specific polymer network formed. The conversion process may involve chemical reactions that enable the oligomer to form cross-linked structures, resulting in a stable network. This network may then be utilized in various applications, potentially offering advantages such as recyclability and reduced environmental impact. The selection of the specific polymer network may depend on the desired properties and intended use of the final product. The process may be designed to ensure that the polymer network retains its recyclability, allowing for repeated use and minimizing waste. This step may highlight the potential for creating sustainable materials through the selective partial depolymerization of natural lignin, aligning with broader goals of environmental responsibility and resource efficiency.

In the context of step 110, the process may involve the simultaneous or sequential execution of photocatalytic activation and the addition of an exogenous electron mediator/scavenger system. The photocatalytic activation may be facilitated by a catalyst, such as tetrabutylammonium decatungstate (TBADT), and a light source emitting ultraviolet light, which may be directed onto the natural lignin source. This activation may target the β-O-4 bond within the lignin structure, potentially leading to its cleavage or modification. The exogenous electron mediator/scavenger system may be introduced to further influence the reaction pathway. The electron mediator may promote the cleavage of the β-O-4 bond, potentially increasing the yield of oligomers while reducing the amount of carbonyl groups. Conversely, the electron scavenger may enhance the oxidation of the β-O-4 bond, potentially increasing the carbonyl content in the resulting oligomers with minimal impact on the yield. The choice between simultaneous or sequential execution of these steps may depend on the desired outcome, such as the balance between oligomer yield and carbonyl content. This approach may offer flexibility in tailoring the depolymerization process to achieve specific characteristics in the resulting oligomers, which may be further processed into chemically recyclable polymer networks.

In the context of the method for selective partial depolymerization of natural lignin, step 112 may involve the performance of photocatalytic activation at a temperature ranging from about 10° C. to about 150° C. This step may ensure that the photocatalytic activation process is conducted under thermal conditions, which may influence the efficiency and effectiveness of the β-O-4 bond activation in the natural lignin source. The temperature range may be selected to facilitate the activation process while maintaining the stability of the catalyst, tetrabutylammonium decatungstate (TBADT), and the integrity of the natural lignin source. The photocatalytic activation may involve the use of a light source emitting ultraviolet light, which may be shined on the natural lignin to provide the energy for the activation of the β-O-4 bond. The temperature control during this process may be essential to balance the reaction kinetics and the thermal stability of the involved components, potentially leading to an efficient depolymerization process. The method may also consider the influence of temperature on the interaction between the catalyst and the lignin source, which may affect the overall yield and quality of the resulting oligomer. The temperature range specified may allow for flexibility in the process, accommodating variations in the natural lignin source and the desired characteristics of the oligomer.

In step 114, the process of photocatalytic activation may be performed for a duration ranging from about 1 hour to about 24 hours. This step may involve the use of a photocatalytic activation system, which may include a catalyst such as tetrabutylammonium decatungstate (TBADT) and a light source emitting ultraviolet light. The purpose of this step may be to facilitate the activation of the β-O-4 bond in the natural lignin source. The duration of the photocatalytic activation may determine the extent of the bond activation and subsequent reactions. The process may be designed to optimize the conditions under which the β-O-4 bond is activated, potentially leading to the desired depolymerization of the lignin. The duration of the activation may be adjusted based on the specific requirements of the process, such as the type of natural lignin source used and the desired properties of the resulting oligomer. The flexibility in the duration of the photocatalytic activation may allow for the fine-tuning of the process to achieve optimal results.

In step 116, the process may involve the addition of an exogenous electron mediator/scavenger system in a specific amount, potentially ranging from about 0.1 mol % to about 10 mol % based on the moles of the β-O-4 bond present in the natural lignin source. This step may be in the selective partial depolymerization of natural lignin, as it may facilitate the cleavage or bond-scission of the β-O-4 bond, which is a structural component in lignin. The exogenous electron mediator may be added to promote the cleavage of the β-O-4 bond, potentially increasing the yield of the oligomer while decreasing the amount of carbonyl (C═O) groups in the oligomer. Conversely, the exogenous electron scavenger may be added to promote the oxidation of the β-O-4 bond, which may increase the amount of carbonyl (C═O) groups in the oligomer and slightly alter the yield of the oligomer. The precise amount of the exogenous electron mediator/scavenger system added may be critical to achieving the balance between oligomer yield and carbonyl group content. This step may be performed in conjunction with other steps in the process, such as photocatalytic activation, to ensure the effective depolymerization of the natural lignin source. The method may allow for the production of oligomers that can be further converted into chemically recyclable polymer networks, thus contributing to the development of sustainable materials.

In the context of step 118, the process may involve the utilization of tetrabutylammonium decatungstate (TBADT) as a catalyst, which may function as an oxidant in the absence of added electron scavengers. This catalyst may have the capability to extract electrons and protons from the natural lignin source, potentially leading to the production of oxidation products. The process may occur even under a pure nitrogen atmosphere, indicating that the TBADT may inherently possess oxidative properties that facilitate the generation of oxidation products, which may account for approximately 19.0% of the reaction outcome. The TBADT's role as an oxidant may be in the selective partial depolymerization of natural lignin, as it may enable the cleavage of specific bonds within the lignin structure, thereby contributing to the formation of desired products. The absence of electron scavengers may imply that the TBADT alone may suffice to drive the oxidation process, highlighting its effectiveness in the depolymerization method. This step may underscore the potential of TBADT to act independently as an oxidizing agent, which may be advantageous in simplifying the reaction setup and reducing the need for additional reagents. The process may be designed to optimize the yield of oxidation products while maintaining the integrity of the lignin structure, thereby achieving a balance between depolymerization and product formation

Providing additional empirical data, the following Examples do not limit the technology and can be inter-combined with any discussion above.

### EXAMPLES

### Example 1. Valorization of Native Lignin

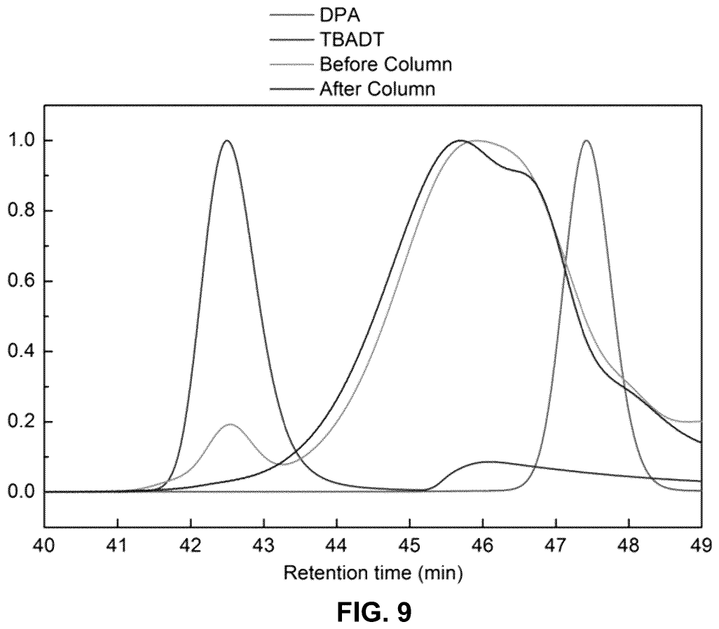

For this purpose, we conducted experiments on pretreated beech sawdust (Fagus sylvatica, Blegwood, for example, catalog 874875223 on etsy.com) following reported procedures with minor modifications. (8,17) Atypical reaction was performed with a mixture of 2.50 g of woodmeal, 75 μmol of TBADT, and 30 mL of 2:1 acetone-acetonitrile mixture, which was then kept at 50° C. using a water bath with irradiation from the same UV LED light as noted above (i.e., 200 W UV LED light centered at ca. 365 nm). The crude oligomers were purified with silica gel and neutral aluminum oxide column chromatography to remove the remaining catalysts (FIG. 9).

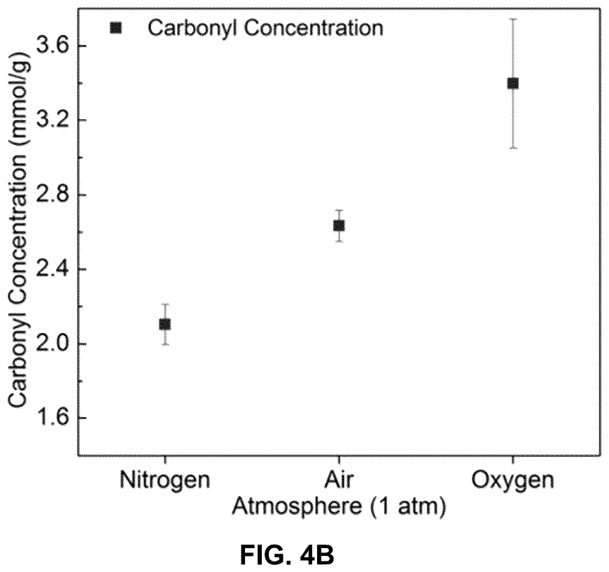

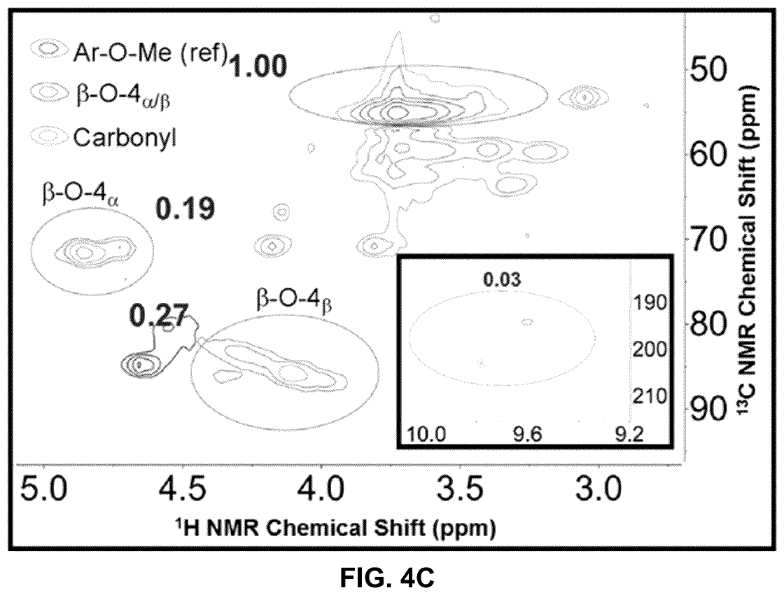

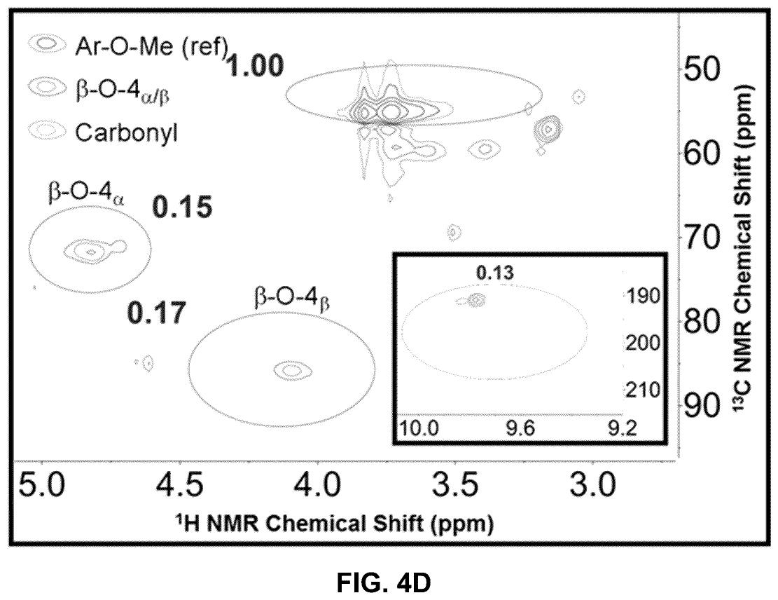

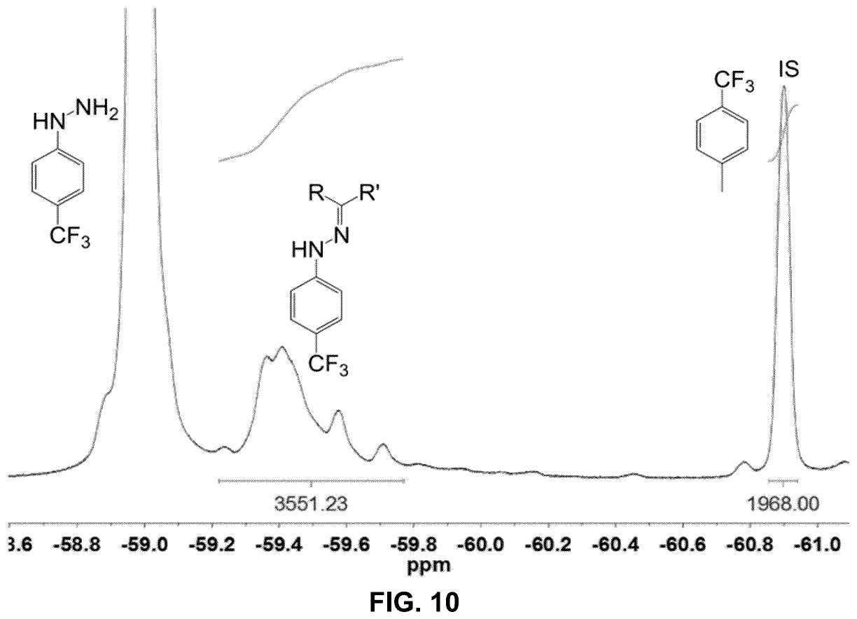

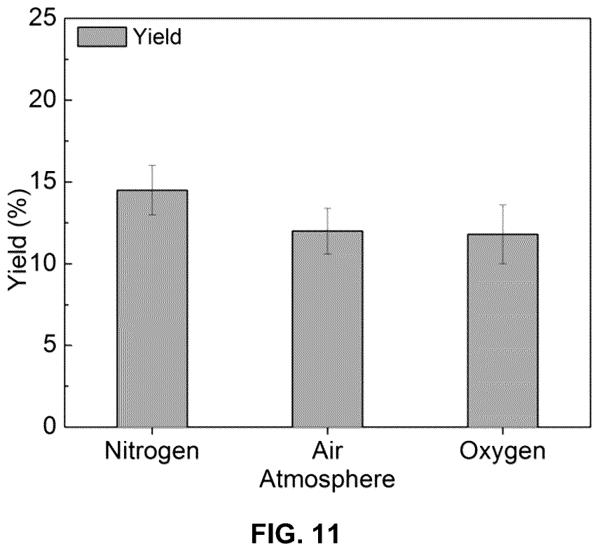

In characterizing the products, attention was focused on soluble oligomer products, and two key figures of merit were quantified, namely, the yield (the weight of oligomer divided by Klason lignin amount in the raw material; (8) see below for more detailed calculations) and the C═O stoichiometry (FIG. 10). With only TBADT present, a yield of 14.5% was obtained, and the products featured a C═O stoichiometry of 2.10 mmol/g. Adding DPA as an electron mediator increased the yield but decreased the C═O stoichiometry, as expected. For instance, with 250 mol % of DPA, the yield was up to 23.3% and the C═O stoichiometry was down to 1.62 mmol/g (FIG. 4A). Conversely, when the reaction was carried out in different atmospheres, the presence of 02 as an electron scavenger resulted in significant increases of the C═O stoichiometry, from 2.10 mmol/g in pure N₂ to 3.40 mmol/g in pure 02 (FIG. 4B), with only modest changes to the yield (FIG. 11). Importantly, the reaction could be performed in ambient air, and low concentrations of water or CO₂ did not appear to influence the process.

FIG. 4A, FIG. 4B, FIG. 4C, and FIG. 4D show examples of photocatalytic conversion of native lignin to oligomers under varying conditions. FIG. 4A shows a comparison of the yield and C═O stoichiometry obtained from photocatalytic degradation of native lignin with varying DPA amount. FIG. 4B shows a comparison of the C═O stoichiometry obtained with varying atmosphere. FIG. 4C shows a 2D-HSQC spectrum of organosolv lignin showing the integration of functional groups of interest. FIG. 4D shows a 2D-HSQC spectrum of lignin oligomers showing the integration of functional groups of interest.

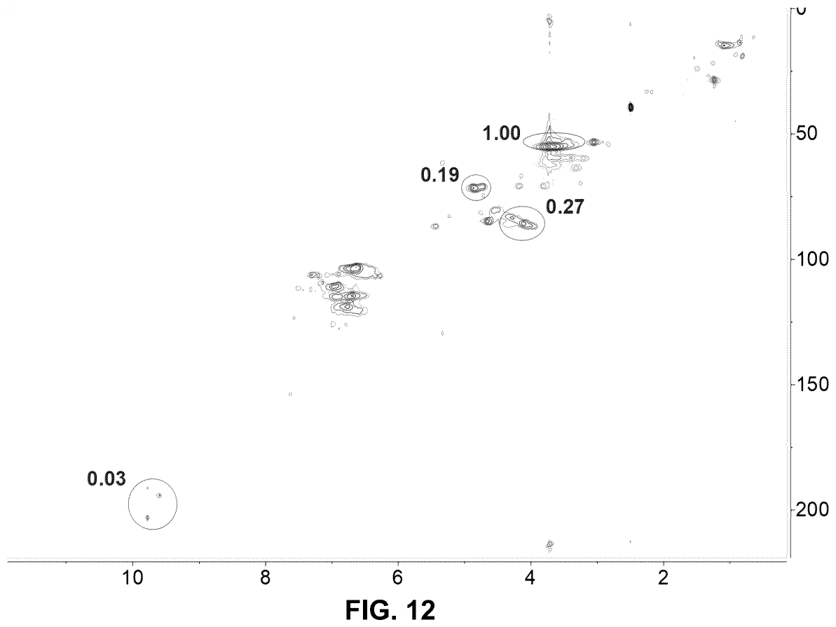

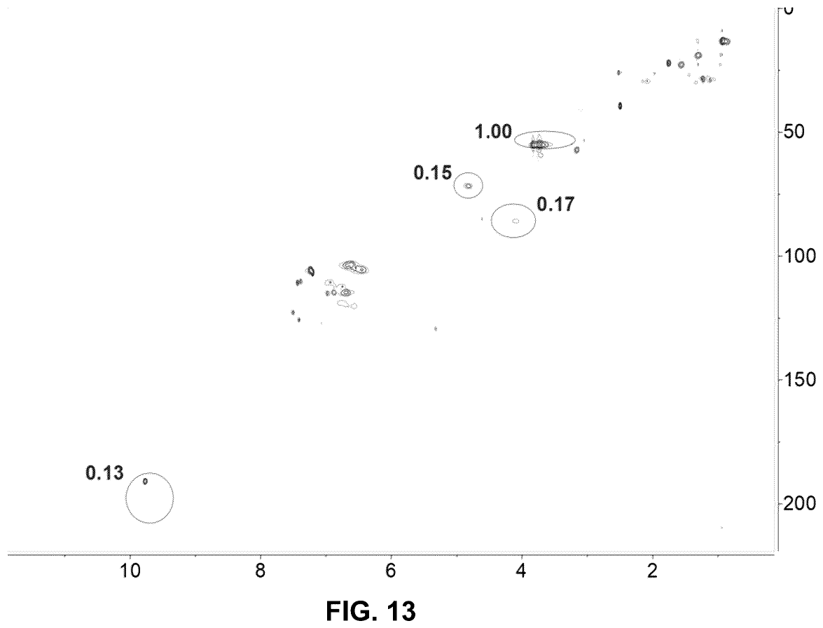

To further confirm that β-O-4 bond cleavage took place, 2-dimensional heteronuclear single-quantum coherence spectroscopy (2D-HSQC) NMR (FIG. 12 and FIG. 13) experiments were conducted using organosolv lignin (47) as a soluble surrogate to native lignin (FIG. 4C and FIG. 4D). The use of organosolv lignin as a soluble surrogate of native lignin is well documented in the literature. (17,22) The 2D-HSQC NMR spectrum of organosolv lignin was consistent with the literature, showing the presence of β-O-4_{α}- and β-O-4_{β}/β′-O-4_{β}-type bonds at ¹H 4.6-5.0 ppm, ¹³C 66.3-76.5 ppm and ¹H 3.8-4.5 ppm, ¹³C 78.9-92.5 ppm, respectively. (47-49) Similar to other reports, (18,47) the aryl methoxy (Ar-OMe) contours (¹H 3.2-4.1 ppm, ¹³C 49.5-56.6 ppm) were used as an internal standard for comparison of the two samples as they remained unchanged by the photocatalytic degradation. A direct comparison was made by setting the integration of the Ar-OMe peak as 1.00 and integrating the same regions of interest in both samples.

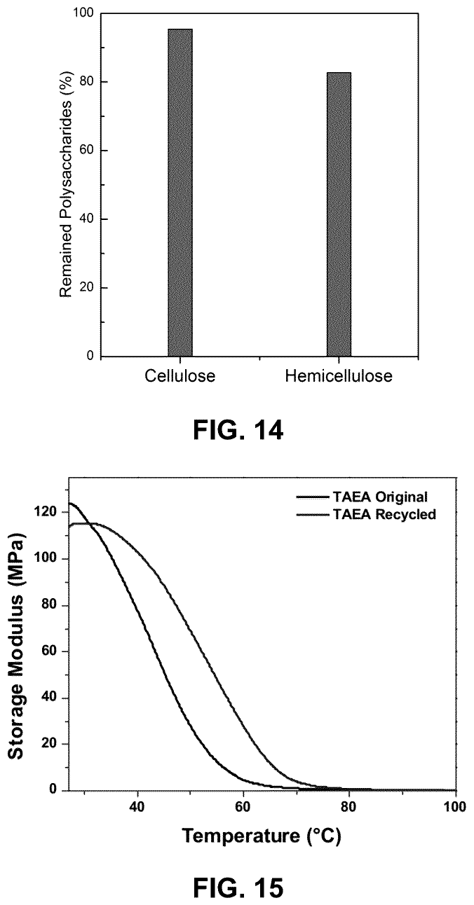

After the photocatalytic degradation of native lignin to oligomers, the integration of the contours of the β-O-4_{α} and β-O-4_{β}/β′-O-4_{β} bonds showed a decrease from 0.19 to 0.15 and from 0.27 to 0.17, respectively, suggesting a partial cleavage of these bonds. In addition, the increase in intensity of the contours in the aldehyde region (¹H 9.3-10.0 ppm, ¹³C 187.5-207.7 ppm) further supports the cleavage of the β-O-4 bond and the generation of the carbonyl functionalities in the resulting oligomers. The evidence strongly supports the proposed mechanisms as shown in FIG. 1C and serves as a foundation to the photocatalytic lignin-first strategy. It is also noteworthy that the chemical integrity of the cellulose and hemicellulose was not affected by our photocatalytic conditions and can be recycled after the reaction (FIG. 14).

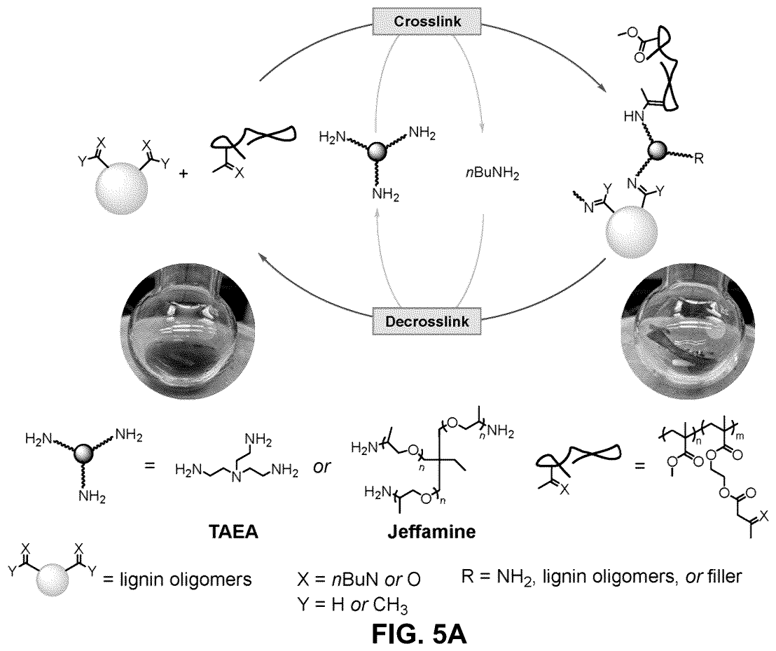

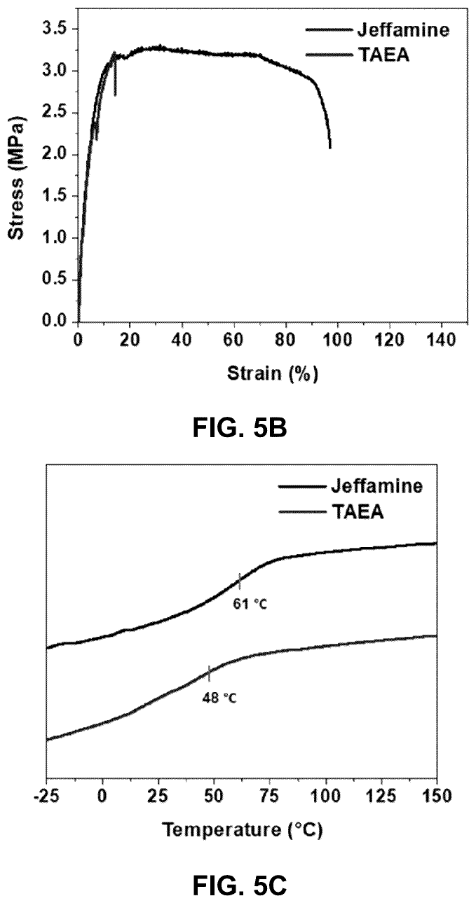

With the ability to obtain lignin oligomers rich in carbonyl functional groups, we set out to create a lignin-based polymer network material by repolymerizing the lignin oligomers with triamine cross-linkers (FIG. 5A); Inspired by the work, (50,51) a copolymer of methyl methacrylate (MMA) and (2-acetoacetoxy)ethyl methacrylate (AAEMA) was used as a chemically recyclable filler to improve the mechanical strength of the network. The polymer network consisted of 40 wt % lignin oligomers and 60 wt % copolymer filler that was first cross-linked by a tris(2-aminoethyl)amine (TAEA) cross-linker. A sub-stoichiometric amount (1 mol % amine groups with respect to total moles of cross-linkable carbonyls) of TAEA was found to be necessary to maintain the dynamic nature of the polymer network and avoid overhardening of the material. After curing at ambient temperature under vacuum for 24 h, tensile test revealed that the cross-linked network was brittle in nature, exhibiting a maximum stress of 3.25 MPa and maximum strain of 15%, likely due to the short nonflexible arms of TAEA (FIG. 5B).

To overcome this challenge, a commercially available trimethylolpropane tris[poly(propylene glycol), amine terminated]ether (Jeffamine) was used as the cross-linker. Jeffamine showed the ability to create a polymer network with improved tensile properties at the stoichiometric amount (100 mol % amine groups with respect to total moles of cross-linkable carbonyls). The maximum stress of the Jeffamine network was similar to that of the TAEA network at ca. 3.25 MPa, while the strain improved to ca. 98% before breaking. The Young's modulus was similar for either the TAEA network or the Jeffamine network at 78 or 76 MPa, respectively. Thermal analysis of the two cross-linked networks by differential scanning calorimetry (DSC) revealed the glass transition temperature (T_{g}) of the TAEA network to be 48° C. and the Jeffamine network to be 61° C. (FIG. 5C). The higher T_{g} of the Jeffamine cross-linked network is likely a result of a higher cross-linking density given that the initial loading of cross-linker was higher. Notably, the polymer network was readily depolymerized into soluble lignin oligomers and the copolymer filler by treating the film with excess n-butylamine at 80° C. Following extraction to remove excess n-butylamine, the residues were repolymerized by simply recurring at ambient temperature followed by drying under vacuum for 24 h. The recurred sample exhibited similar mechanical properties as the original network (FIG. 15). Overall, we demonstrated that the lignin oligomers generated from the catalytic depolymerization of native lignin can be repolymerized into mechanically robust polymer networks capable of closed-loop chemical recycling. FIG. 5A shows a schematic illustration of the formation and recycling of lignin oligomer containing DPNs. FIG. 5B shows tensile tests comparing different triamine cross-linked networks. FIG. 5B shows glass transition temperatures of cross-linked networks as determined by DSC cooling curves.

We have developed a new approach to the direct conversion of native lignin to oligomers rich in carbonyl functionalities under photocatalytic conditions. The products were then used to prepare closed-loop, chemically recyclable DPNs. Toward this goal, TBADT as a photocatalyst made of earth-abundant elements was shown to be effective in either cleaving or oxidizing β-O-4 motifs through a HAT mechanism under conditions that would preserve cellulose and hemicellulose. The selectivity between decomposition and oxidation was controlled by adding exogenous electron mediators or electron scavengers. When applied to native lignin, the photocatalytic approach was shown to produce oligomers with up to 23.3% yield. The carbonyl stoichiometry was between 1.62 and 3.40 mmol/g, depending on the reaction conditions. 2D-HSQC results further supported that our photocatalytic system partially consumed β-O-4 motifs and generated high-value carbonyl functionalities. The produced oligomers could be repolymerized to form chemically recyclable polymer networks via imine bond formation. The DPNs were capable of closed-loop recycling. The present work not only represents a new strategy for lignin valorization under mild conditions beyond monomerization but also provides the ability to generate closed-loop recyclable lignin-based materials with promising material properties.

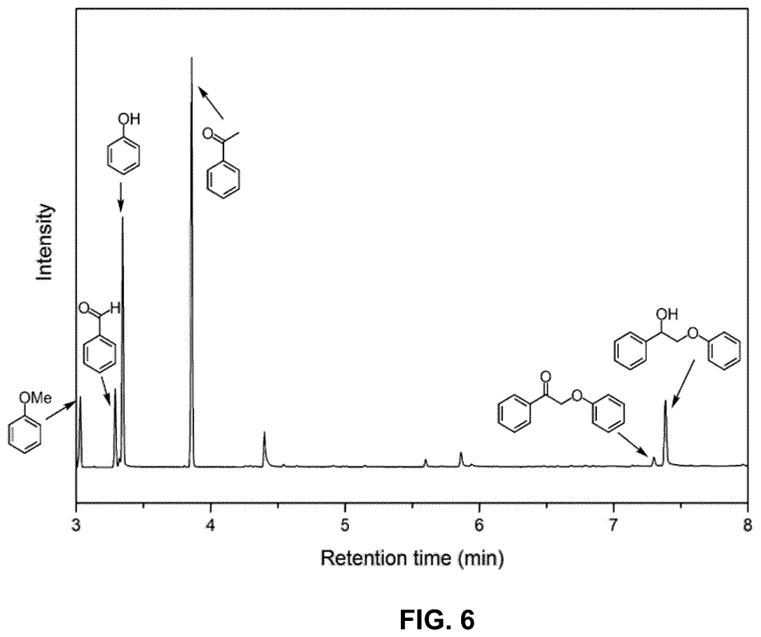

The results and methods described herein are including general experimental information and experimental details, 1H and 2D-HSQC NMR characterization, SEC traces, Klason lignin information, carbonyl quantification experiments, summary of PPol photocatalysis under varying conditions, summary of PPol vs PPdiol photocatalysis products, and summary of native lignin photocatalysis results under varying conditions (PDF), such that a person of skill can repeat the claimed procedures. No unexpected or unusually high safety hazards were encountered. The methods are scalable and can be continuous. FIG. 6 shows a gas chromatograph of the photocatalytic degradation of PPol. FIG. 7 shows a comparison of the products of the photocatalysis of PPol using varying amounts of TBADT. FIG. 8 shows a gas chromatograph of the photocatalytic degradation of PPol under 100% oxygen atmosphere. FIG. 9 shows an SEC of catalysts, crude oligomers, and purified oligomers.

### Klason Lignin Information

Klason lignin content was measured according to a literature procedure in order to calculate the yield of lignin oligomers.⁽⁵²⁾ In a typical experiment, woodmeal (0.2 g) and 72% sulfuric acid (1.5 mL) were mixed in a glass vial and stirred for 1 hour at 30° C. in a water bath. The mixture was diluted to 42 mL with DI water and transferred to an autoclave reactor. The solution was heated to 121° C. for 1 hour. After cooling to room temperature, the brown precipitate (Klason lignin) was separated by filtration. The solid was washed with DI water (3×20 mL). The Klason lignin was dried at 105° C. for 24 hours to afford the product (16.1±0.1%).

$$Klason lignin content ( \% ) = \frac{Klason lignin product ( g )}{Total biomass weight ( g )} \times 100 \%$$

### Carbonyl Quantification Experiments

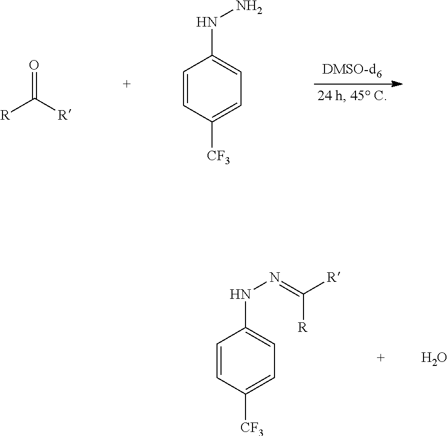

To calculate the amount of carbonyls in the lignin oligomers, a published method about using 4-(trifluoromethyl)phenylhydrazine to derivatize carbonyls to the corresponding hydrazone was applied.⁽⁵³⁾ For a typical quantification reaction, lignin oligomers (15 mg), 100 μL p-trifluoromethyl toluene (Internal standard, IS) in DMSO-d6 solution (0.1682 μmol/mg, accurately weighed), 200 μL 4-(trifluoromethyl)phenylhydrazine in DMSO-d6 solution (containing 70.5 mg 4-(trifluoromethyl)phenylhydrazine, 0.40 mmol), and 400 μL pure DMSO-d6 were mixed in a 4 mL vial, and then homogenized in a vortex mixer. The mixture was then transferred to an NMR tube and heated to 45° C. for 24 hours. Before NMR analysis, 100 μL of relaxation agent solution in DMSO-d6 (containing 2.8 mg chromium acetylacetonate) was added and homogenized. 19F NMR was carried out with the following acquisition parameters: 90° pulse angle, relaxation delay=3 sec, scans=64, region=−50-70 ppm. IS was used as a reference peak set to −60.9 ppm, with the unreacted hydrazine peak at −59 ppm.

The total amount of carbonyls present is calculated as follows:

### Molarity of IS

$$117. mg \times 0.1682 μmol / mg = 19.68 μmol Internal standard molecules$$

### Concentration of Carbonyls in Sample

$$35.51 μmol / 13.2 mg = 2.69 mmol / g$$

For example, FIG. 10 shows an NMR of the hydrzaone formation for the quantification of carbonyl concentration in DMSO-d6 600 MHz. FIG. 11 shows a comparison of the yields of the photocatalysis of native lignin to lignin oligomers under varying atmospheres. FIG. 12 shows a full 2D-HSQC of organosolv lignin in DMSO-d6, 500 MHz. FIG. 13 shows a full 2D-HSQC of lignin oligomers in DMSO-d6, 500 MHz. FIG. 14 shows a quantification of remaining cellulose and hemicellulose after photocatalysis. FIG. 15 shows a DMA analysis of original crosslinked TAEA network and recycled TAEA network.

Materials: All reactions were carried out under a nitrogen atmosphere unless otherwise stated. Nitrogen and oxygen gasses were purchased from airgas. For some examples, beech wood sawdust was purchased from Blegwood. All solvents including ethanol, acetone (AC), anhydrous acetonitrile (AN), dichloromethane (DCM), dioxane, ethyl acetate (EA), hexane (Hex), methanol (MeOH), and tetrahydrofuran (THF) were purchased from commercial sources and used without further purification unless otherwise stated. Dioxane was further dried with a drying column of a solvent system from Pure Process Technologies. NMR solvents including dimethyl sulfoxide (DMSO-d6) and chloroform (CDCl3) were purchased from Cambridge Isotope Laboratories or ACROS organics.

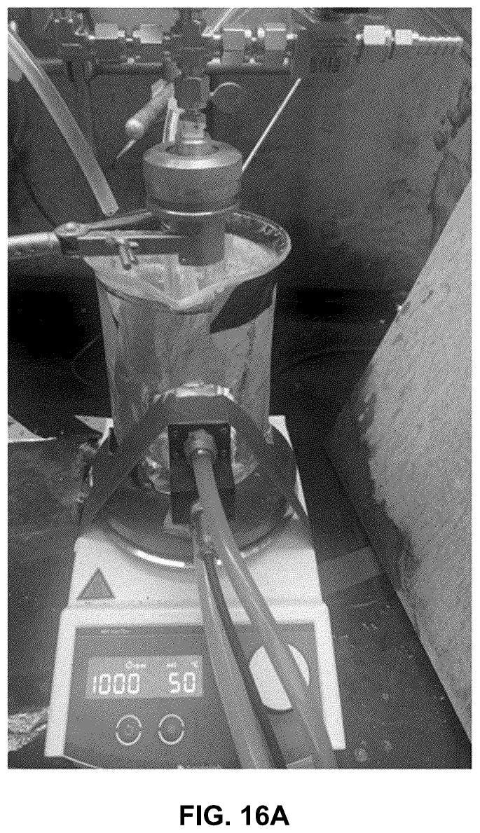

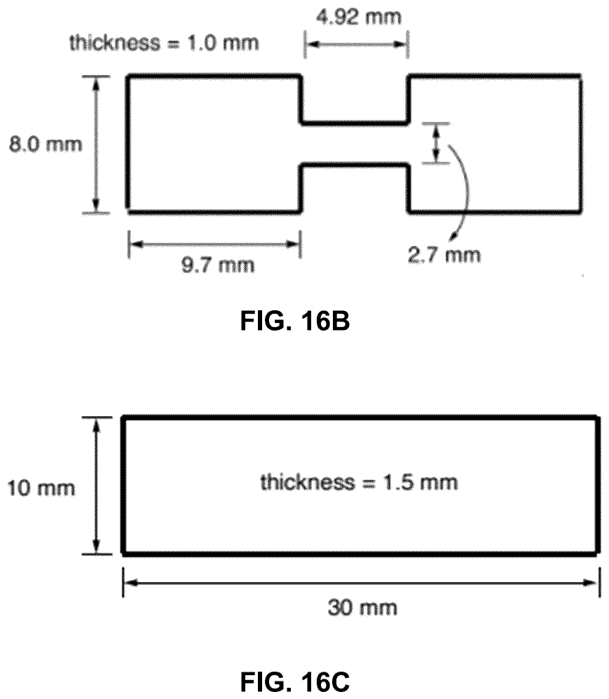

Sodium tungstate dihydrate and tetrabutylammonium bromide (TBAB) were purchased from Acros organics. Diphenyl acthracene (DPA) was purchased from Alfa Aesar. 2-phenoxy-1-phenylethanol (PPol) and 4-Cyano-4-(dodecylsulfanylthiocarbonyl)sulfanylpentanoic acid were purchased from Ambeed. Methyl 2-methylprop-2-enoate (MMA) and n-butylamine were purchased from TCI. (2-acetoacetoxy)ethyl methacrylate (AAEMA), azobisisobutyronitrile (AIBN) (recrystallized from methanol prior to use), cellulose, hemicellulose, 4-(trifluoromethyl)phenylhydrazine, trimethylolpropane tris[poly(propylene glycol), amine terminated]ether (Jeffamine), and tris(2-aminoethyl)amine (TAEA) were purchased from Sigma Aldrich. MMA and AAEMA were passed through a basic aluminum oxide plug prior to use. SiliaFlash (P60 40-63 μM (230-400 mesh)) silica gel was purchased from SiliCycle and used for column chromatography. Basic aluminum oxide (Brockmann 1 50-200 μm, 60A) was used for reagent purification. Neutral aluminum oxide (150 mesh) was used for oligomer purification. Thin Layer Chromatography (TLC) Silica Gel 60 F254: Glass Plates 20×20 cm were used for TLC analysis. The polytetrafluoroethylene (PTFE) mold used for creating tensile test and DMA samples had the following measurements, respectively, shown in FIG. 16B and FIG. 16C, which show the molds (example dimension).

Characterization: All 1H, 13C, and 19F NMR spectra were obtained using a Varian 600 MHz, Varian 500 MHz, Bruker 500 MHz equipped with a X nuclei optimized 5 mm double resonance BBO H&F CryoProbe Prodigy, or Bruker 500 MHz equipped with a CryoProbe (He) BBFO 5 mm BBF/1H X nuclei optimized 5 mm double resonance BBFO, NMR spectrometer. Size exclusion chromatography (SEC) was carried out on a Tosoh's high-performance SEC system HLC-8320GPC containing TSKgel Alpha-M columns at 50° C. and a flow rate of 0.6 mL/min. The eluent was HPLC grade DMF with 0.01 M LiBr.

Polystyrene standards (ReadyCal Kit, Sigma-Aldrich #81434) were used to determine the molecular weight and molecular weight distribution of the polymers. Prior to injection into the SEC, polymers were dissolved in DMF and filtered through a 0.20 μm PTFE filter. Differential scanning calorimetry (DSC) was performed using Tzero hermetic pans and lids on a TA Instrument Discovery DSC 250. Scans were performed from −50-200° C., over three cycles at a rate of 10° C./min. An isotherm step for 5 minutes was used at both −50 and 200° C. Tensile test was performed on an Instron 5543 universal testing system that was equipped with a 100 N load cell. The strain rate was set at 2 strain/min. Dynamic mechanical analysis (DMA) was carried out on a TA Instruments Discovery Dynamic Mechanical Analyzer 850.

### Example Light Reactor Specifications and Setup:

The light source was a 200 W UV LED light centered at 365 nm, from Howsuper, with the source number H6015-S-6868-LG-365 nm. FIG. 16A shows an example light setup used for photocatalysis.

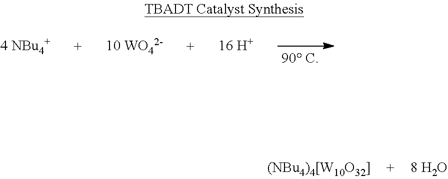

TBADT catalyst was synthesized according to a literature procedure with minor modifications.(54) TBAB (4.8 g, 15 mmol) and Na2WO4·2H2O (10.0 g, 30 mmol) were dissolved in separate deionized (DI) water (50 mL and 100 mL, respectively) solutions. The solutions were acidified to pH 2 with concentrated hydrochloric acid. After heating to 90° C., the solutions were mixed together. Precipitation was observed immediately, indicating the formation of TBADT. The slurry was stirred for 30 minutes in a 90° C. water bath, cooled to room temperature, and filtered with a Büchner funnel. The solid phase was washed with DI water and THF (3×30 mL) and dried in a vacuum oven at 90° C. overnight. The crude TBADT was further purified by recrystallization. The crude TBADT was refluxed in DCM (1 g: 20 mL) for 2 hours. The mixture was cooled on an ice bath, and then filtered to obtain pure TBADT as a transparent crystal with a light-yellow color (4.02 g, 40.0% based on W).

Photocatalysis of PPol or PPdiol without DPA

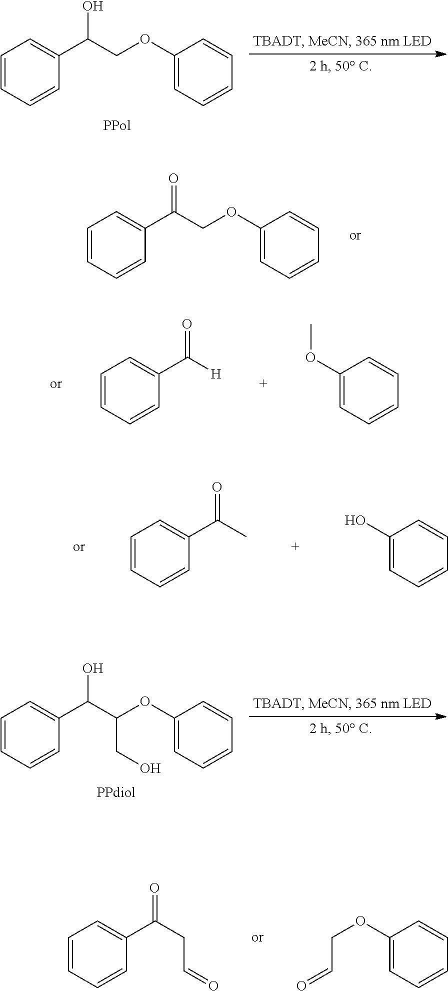

For a typical photocatalytic reaction of PPol or PPdiol without DPA, PPol (10.7 mg, 50 μmol) or PPdiol (12.2 mg, 50 μmol), TBADT (5.3 mg, 1.6 μmol), and AN (1 mL) were added to a 10 mL Schlenk tube. The solution was degassed by the freeze-pump-thaw method (3×) such that the atmosphere of the Schlenk tube was 1 atm nitrogen. The solution was then heated to 50° C. in a water bath and irradiated with UV light for 2 hours. The products were then analyzed and quantified by 1H NMR (48.0% conversion, 9.1% PPone, 29.2% acetophenone, 9.7% benzaldehyde). See table shown in FIG. 17A for yield details.

Photocatalysis of PPol with DPA: The procedure is the same as the photocatalysis of PPol without DPA. DPA amounts used were as follows: 66.1 μg, 0.2 μmol or 220 μg, 0.67 μmol or 661 μg, 2.0 μmol See Table S1 for yield details. Photocatalysis of PPol with Oxygen:Nitrogen Mixture Atmosphere Varying oxygen mixtures were prepared by mixing pure oxygen and pure nitrogen in a high-pressure reactor. For 2%, 5% and 20% oxygen, the partial pressures for oxygen and nitrogen were 0.5 bar/24.5 bar, 0.5 bar/9.5 bar, 2 bar/8 bar, respectively. The procedure is the same as the photocatalysis of PPol reaction without DPA; however, the oxygen:nitrogen gas mixtures were introduced instead of pure nitrogen during freeze-pump-thaw cycles. Oxygen percentages used are as follows: 2% oxygen or 5% oxygen or 20% oxygen See Table S1 for yield details.

FIG. 17A shows a table with a summary of PPol photocatalysis under varying conditions. FIG. 17B shows a table with a summary of PPol vs. PPdiol photocatalysis products.

Photocatalysis of Native Lignin without DPA For a typical photocatalytic reaction of native lignin, woodmeal (2.50 g), TBADT (250 mg, 75 μmol), and AC:AN (30 mL 2:1) were mixed together in a 3 oz pressure reaction vessel. The system was degassed by freeze-pump-thaw in the same manner as the photocatalysis of PPol. The reactor was heated in a water bath to 50° C. and stirred for 48 hours under UV light irradiation. The oligomer solution was then collected by filtering the resulting slurry. The solution was concentrated under reduced pressure and the residue was resuspended in THF and sonicated for 5 minutes. The solution was then passed through a syringe filter (PET-45/25 polyester membrane with pore diameter of 0.45 μm, and membrane diameter of 25 mm) to remove the precipitated TBADT. The crude oligomers were purified with silica gel column chromatography. A mixture of hexane and ethyl acetate (8:2) was used to remove non-polar impurities. The gradient was then switched to DCM and methanol (1:0-0:1) to obtain the remaining oligomers. The collected oligomers were then subjected to a neutral aluminum oxide plug (DCM:MeOH 1:0-0:1) to further purify the oligomers (58.2 mg, 14.5%). See Table in FIG. 17B for yield details.

Photocatalysis of Native Lignin with DPA: The procedure is the same as the photocatalysis of native lignin without DPA. DPA amounts used were as follows: 20.7 mg, 62.5 μmol or 41.3 mg, 125 μmol S14 or 62.0 mg, 187.5 μmol See Table in FIG. 17B for yield details.

Photocatalysis of Native Lignin with Different Atmospheres: The procedure is the same as the photocatalysis of native lignin without DPA; however, either oxygen gas or air (1 atm) were introduced instead of pure nitrogen during freeze-pump-thaw cycles. See FIG. 17B for yield details.

FIG. 17C shows a table with a summary of native lignin photocatalysis results under varying conditions.

Preparation of Organosolv Lignin Organosolv: lignin for 2D-HSQC NMR was synthesized according to the literature.4 Pretreated woodmeal (5 g), 80% ethanol aqueous solution (40 mL), and concentrated hydrochloric acid (0.8 mL) were added to a 100 mL round-bottom flask. The mixture was refluxed at 80° C. for 5 hours at which point the mixture was filtered. The residue was washed with 100% ethanol (4×5 mL). The filtrate was collected and concentrated under reduced pressure. The remaining solid from the filtrate was re-dissolved in acetone (5 mL). The solution was then precipitated into DI water by dropwise addition (100 mL). Upon addition to the water, a brown-pink precipitate formed. The solution was filtered and the collected solid was dried in a vacuum oven at 45° C. overnight to afford the product (0.25 g, 5%).

2D-HSQC NMR Experimental Details: To obtain 2D-HSQC spectra of organosolv lignin and the generated lignin oligomers, 25 mg of each sample was dissolved in 600 μL of DMSO-d6. The spectra were run on a Bruker 500 MHz equipped with a CryoProbe (He) BBFO 5 mm BBF/1H X nuclei optimized 5 mm double resonance BBFO. The pulse sequence selected was hsqcedetgpsisp2.⁽⁵⁴⁾ The acquisition parameters were as follows: NS=8, AQ=0.08704 sec, D1=1 sec, 1SW=220 ppm, O1P=6.000, 2SW=11.7592, O2P=110.00, DB=0, 1TD=256, 2TD=1024. The data was processed using MestReNova x64-14.1.1-24571. DMSO-d6 was used as a reference peak set to 1H 2.50 ppm, 13C 39.52 ppm. The raw data was auto phase corrected, and auto baseline corrected before integration. The contours were integrated using the following regions: Ar-OMe (1H 3.2-4.1 ppm, 13C 49.5-56.6 ppm) (internal standard), β-O-4_{α} (1H 4.6-5.0 ppm, 13C 66.3-76.5 ppm), βO-4β/β′-O-4β (1H 3.8-4.5 ppm, 13C 78.9-92.5 ppm), aldehyde (1H 9.3-10.0 ppm, 13C 187.5-207.7 ppm).

Wood Sawdust (Native Lignin) Pretreatment: Beech wood sawdust was pretreated according to a literature procedure with minor modifications.⁽⁵⁶⁾ The wood sawdust (1 g:10 mL) was suspended in an ethanol-water solution (4:1) and sonicated for 2 hours to remove most of the extractives. The solution was filtered, and the solid was washed with acetone (3×100 mL), and dried in a vacuum oven at 45° C. for a minimum of 48 hours. The solid was then oven-dried in 2 hour intervals at 105° C. until the weight change was <5% indicating a <5% moisture content. The dry sawdust was then knife-milled through a 1 mm screen. The wood powder was sealed and stored at 0° C.

### Example 2. Control Experiments

Control Experiments with Cellulose and Hemicellulose: The photocatalysis on cellulose and hemicellulose featured the same conditions and additives as typical woodmeal photocatalysis above, with the only difference being the reactant: using pure cellulose or hemicellulose (1.00 g) instead of woodmeal (2.50 g). After the reaction, the solution was filtered and washed with AN (3×5 mL), to remove TBADT, dried in vacuum oven overnight, and weighed to calculate the remaining percentage (weight after reaction weight before reaction) of each species. After photocatalysis, cellulose (95.4%) and hemicellulose (82.7%) were recovered. See FIG. 14. Synthesis of Polymer Filler

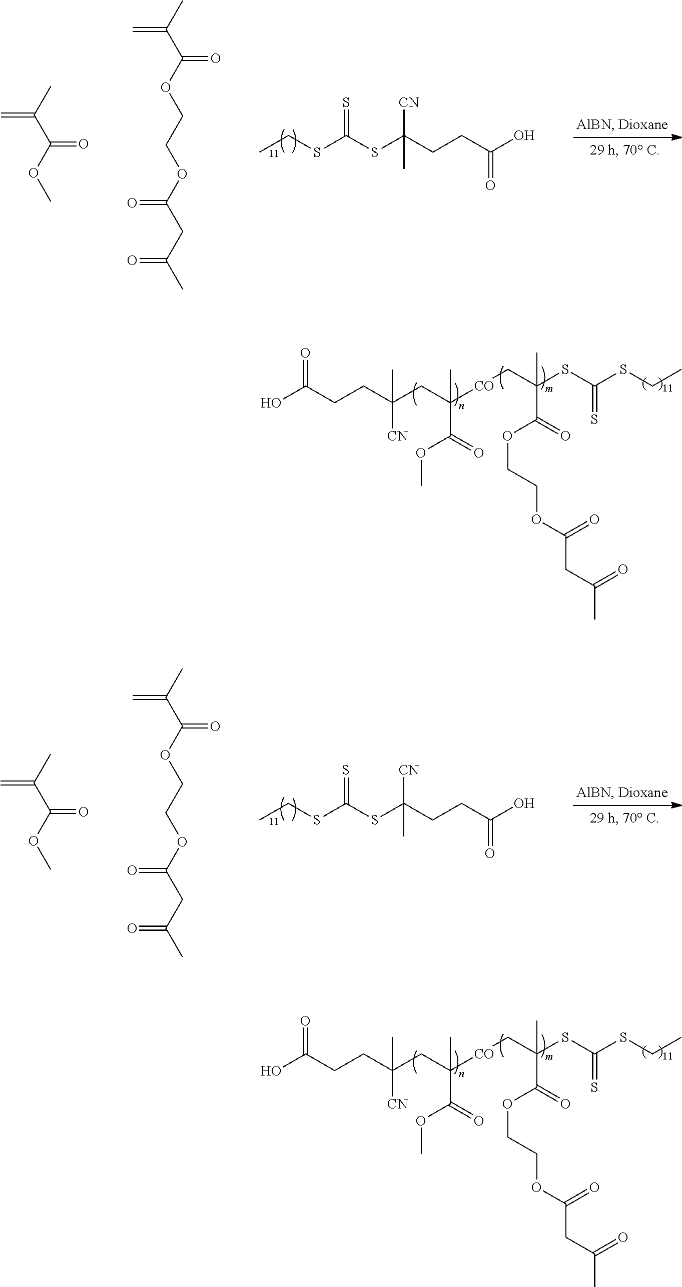

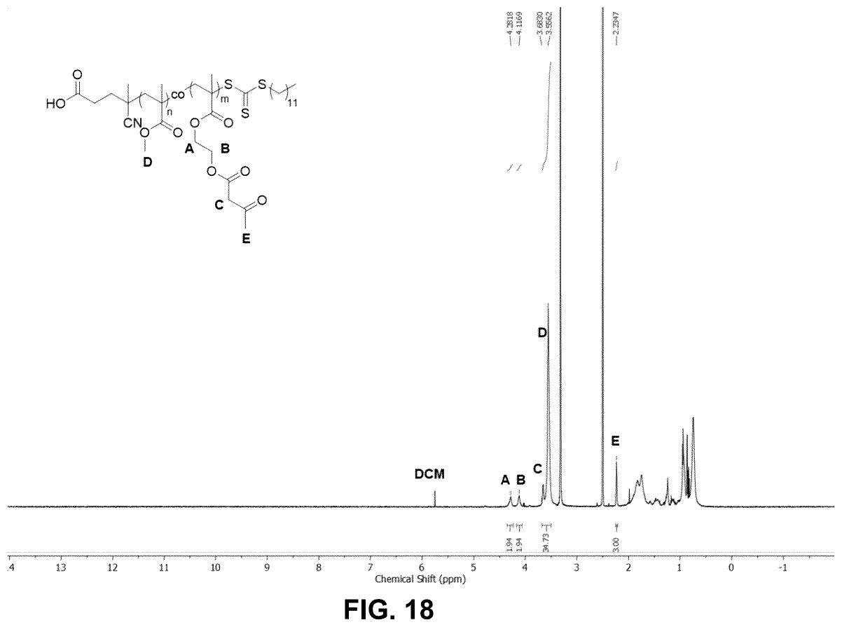

MMA (2.00 mL, 18.76 mmol) and AAEMA (358.14 μL, 1.88 mmol) were added to a flask and dissolved in dioxane (15 mL). AIBN (4.62 mg, 28.14 μmol) was added as a dioxane (1 mL) solution. The system was sealed with a septum and subjected to 3 rounds of freeze-pump-thaw to remove oxygen. The solution was heated to 70° C. for 29 hours at which point the solution was concentrated by half and precipitated into hexane (3×45 mL). The product was obtained as a yellow solid (2.00 g). The NMR spectrum is consistent with the literature6 and is shown below (FIG. 18) for the calculation of AAEMA incorporation.

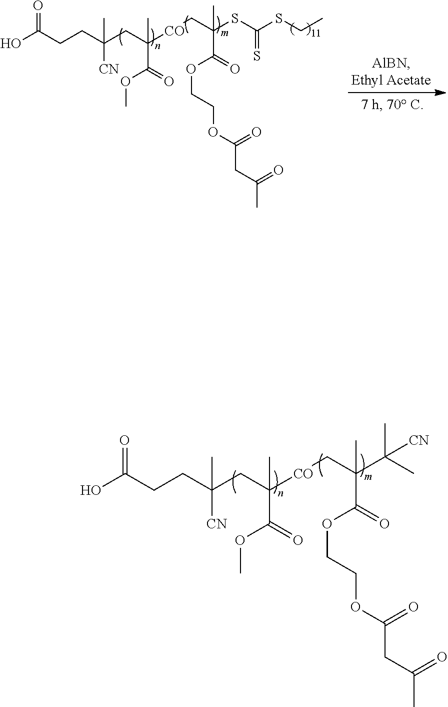

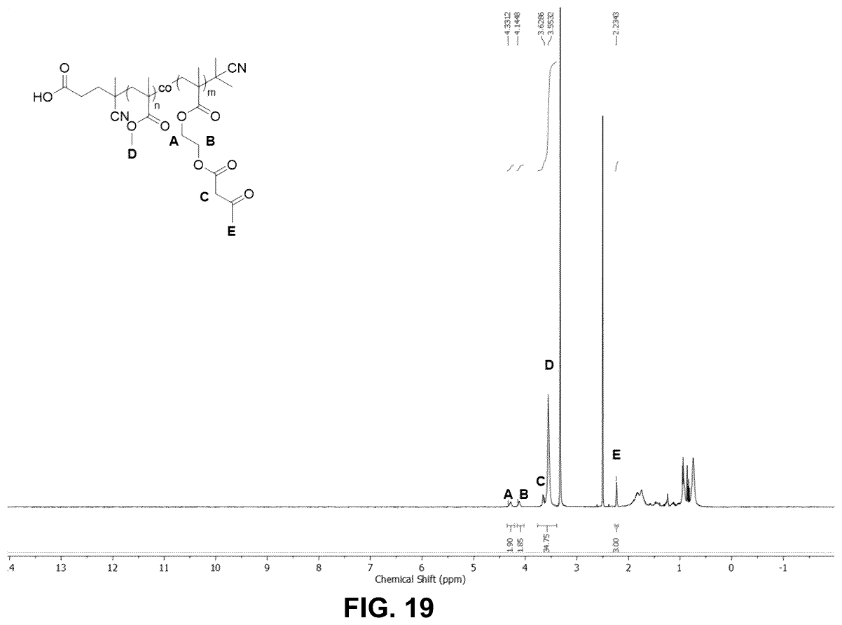

4-cyano-8-dodecylsulfanylcarbothioylsulfanyl-6-methoxycarbonyl-4,6,8-trimethyl-9-oxo-10-[2-(3-oxobutanoyloxy)ethoxy]decanoic acid (2.00 g, 2.73 mmol) and AIBN (30.81 mg, 187.6 μmol) were dissolved in EA (27 mL). The system was degassed with nitrogen for 25 minutes at which point the system was sealed with a septum and heated to 70° C. for 7 hours. The solution was concentrated by half and precipitated into hexane (3×45 mL). The product was obtained as a white solid (1.85 g). The NMR spectrum is consistent with the literature6 and is shown (FIG. 19) for the calculation of AAEMA incorporation.

### TAEA Crosslinking Procedure:

As an example, lignin (34 mg, f=2.892 mmol/g), and copolymer (52 mg, Mn=17.6 kDA) were dissolved in THF (0.296 mL) in a 1.5 mL vial. From a stock solution of TAEA (10 μL in 990 μL of THF) was transferred (6.78 μL, 4.53×10-4 mmol) to the lignin solution. The solution was mixed in a circular motion and transferred within 10 seconds to a PTFE mold via pipette, in order to avoid cross-linking in the mixing vessel. Any air bubbles were carefully removed with the tip of a needle. The solution was allowed to sit at ambient conditions and undergo solvent evaporation for 18 hours at which point the film was carefully removed from the mold. The sample was cured under vacuum at room temperature for 24 hours to remove residual solvents. Jeffamine Crosslinking Procedure: As an example, lignin (34 mg, f=2.800 mmol/g), and copolymer (52 mg, Mn=17.6 kDA) were dissolved in THF (0.250 mL) in a 1.5 mL vial. From a stock solution of Jeffamine (200 μL in 300 μL of THF) was transferred (44.89 μL, 4.0×10-2 mmol) to the lignin solution. The solution was mixed in a circular motion and transferred within 10 seconds to a PTFE mold via pipette, in order to avoid cross-linking in the mixing vessel. Any air bubbles were carefully removed with the tip of a needle. The solution was allowed to sit at ambient conditions and undergo solvent evaporation for 18 hours at which point the film was carefully removed from the mold. The sample was cured under vacuum at room temperature for 24 hours to remove residual solvents. TAEA Recycling Procedure The obtained film was suspended in n-butylamine (4 mL) and heated to 80° C. until all solid was dissolved (4-18 hours). The solution was concentrated under reduced pressure. The residue was then subjected to the initial crosslinking conditions. Jeffamine Recycling Procedure: The obtained film was suspended in n-butylamine (4 mL) and heated to 80° C. until all solid was dissolved (4-18 hours). The solution was partially concentrated to remove about half of the n-butylamine. DCM and water (20 mL) were added. The aqueous phase was extracted with DCM (3×20 mL). The combined organic phases were washed with water (5×100 mL). The combined organic phases were dried over anhydrous sodium sulfate, filtered, and concentrated under reduced pressure. The residue was then subjected to the initial crosslinking conditions.

### Example 3. Examples of Characterizations (NMR and SEC)

Measurement conditions are discussed above.

FIG. 18 shows a 1H NMR of copolymer filler before end group exchange in DMSO-d6, 600 MHz. FIG. 19 shows a 1H NMR of copolymer filler after end group exchange in DMSO-d6, 600 MHz.

AAEMA Incorporation Calculation: To calculate the percentage of AAEMA incorporation, the equation from Sumerlin and coworkers was used.⁽⁵⁷⁾

$$I = integration of peak$$

$$\% AAEMA = \frac{{I}_{e}}{( {I}_{c + d} ) - 2 + 3} \times 100 \%$$

$$\% AAEMA = \frac{3}{34.8 - 2 + 3} \times 100 \% = 8.4 \% incorporation before and after chain end exchange$$

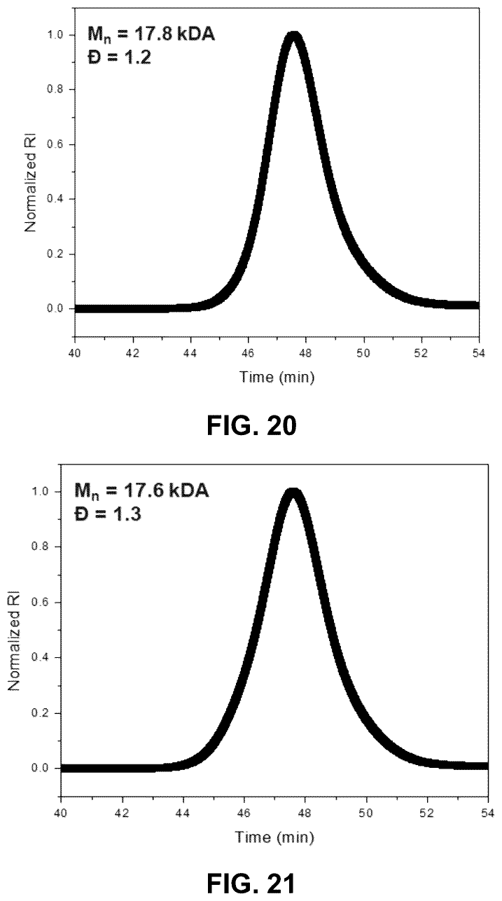

FIG. 20 shows an SEC trace of copolymer filler before end group exchange. FIG. 21 shows an SEC trace of copolymer filler after end group exchange.

### REFERENCES

(1) Schutyser, W.; Renders, a. T.; Van den Bosch, S.; Koelewijn, S.-F.; Beckham, G.; Sels, B. F. Chemicals from lignin: an interplay of lignocellulose fractionation, depolymerisation, and upgrading. Chem. Soc. Rev. 2018, 47 (3), 852-908.
(2) Wu, X.; Luo, N.; Xie, S.; Zhang, H.; Zhang, Q.; Wang, F.; Wang, Y. Photocatalytic transformations of lignocellulosic biomass into chemicals. Chem. Soc. Rev. 2020, 49 (17), 6198-6223.
(3) Huang, Z.; Luo, N.; Zhang, C.; Wang, F. Radical generation and fate control for photocatalytic biomass conversion. Nat. Rev. Chem. 2022, 6 (3), 197-214.
(4) Galkin, M. V.; Samec, J. S. Lignin valorization through catalytic lignocellulose fractionation: a fundamental platform for the future biorefinery. ChemSusChem 2016, 9 (13), 1544-1558.
(5) Sadeghifar, H.; Ragauskas, A. Perspective on technical lignin fractionation. ACS Sustain. Chem. Eng. 2020, 8 (22), 8086-8101.
(6) Eraghi Kazzaz, A.; Fatehi, P. Technical lignin and its potential modification routes: A mini-review. Ind. Crops Prod. 2020, 154, 112732.
(7) Renders, T.; Van den Bosch, S.; Koelewijn, S.-F.; Schutyser, W.; Sels, B. Lignin-first biomass fractionation: the advent of active stabilisation strategies. Energy Environ. Sci. 2017, 10 (7), 1551-1557.
(8) Abu-Omar, M. M.; Barta, K.; Beckham, G. T.; Luterbacher, J. S.; Ralph, J.; Rinaldi, R.; Romen-Leshkov, Y; Samec, J. S.; Sels, B. F.; Wang, F. Guidelines for performing lignin-first biorefining. Energy Environ. Sci. 2021, 14 (1), 262-292.
(9) Jang, J. H.; Brandner, D. G.; Dreiling, R. J.; Ringsby, A. J.; Bussard, J. R.; Stanley, L. M.; Happs, R. M.; Kovvali, A. S.; Cutler, J. I.; Renders, T. Multi-pass flow-through reductive catalytic fractionation. Joule 2022, 6, 1859.
(10) Liu, Z.; Li, H.; Gao, X.; Guo, X.; Wang, S.; Fang, Y.; Song, G. Rational highly dispersed ruthenium for reductive catalytic fractionation of lignocellulose. Nat. Commun. 2022, 13 (1), 4716.
(11) O'Dea, R. M.; Pranda, P. A.; Luo, Y; Amitrano, A.; Ebikade, E. O.; Gottlieb, E. R.; Ajao, O.; Benali, M.; Vlachos, D. G.; lerapetritou, M.; et al. Ambient-pressure lignin valorization to high-performance polymers by intensified reductive catalytic deconstruction. Sci. Adv. 2022, 8 (3), No. eabj7523.
(12) Liao, Y; Koelewijn, S.-F.; Van den Bossche, G.; Van Aelst, J.; Van den Bosch, S.; Renders, T.; Navare, K.; Nicolai, T.; Van Aelst, K.; Maesen, M.; et al. A sustainable wood biorefinery for low-carbon footprint chemicals production. Science 2020, 367 (6484), 1385-1390.
(13) Shuai, L.; Amiri, M. T.; Questell-Santiago, Y M.; Heroguel, F.; Li, Y; Kim, H.; Meilan, R.; Chapple, C.; Ralph, J.; Luterbacher, J. S. Formaldehyde stabilization facilitates lignin monomer production during biomass depolymerization. Science 2016, 354 (6310), 329-333.
(14) Guo, H.; Zhang, B.; Li, C.; Peng, C.; Dai, T.; Xie, H.; Wang, A.; Zhang, T. Tungsten carbide: a remarkably efficient catalyst for the selective cleavage of lignin C— O bonds. ChemSusChem 2016, 9 (22), 3220-3229.
(15) Zaheer, M.; Kempe, R. Catalytic hydrogenolysis of aryl ethers: a key step in lignin valorization to valuable chemicals. ACS Catal. 2015, 5 (3), 1675-1684.
(16) Van Aelst, K.; Van Sinay, E.; Vangeel, T.; Cooreman, E.; Van den Bossche, G.; Renders, T.; Van Aelst, J.; Van den Bosch, S.; Sels, B. Reductive catalytic fractionation of pine wood: elucidating and quantifying the molecular structures in the lignin oil. Chem. Sci. 2020, 11 (42), 11498-11508.
(17) Nguyen, S. T.; Murray, P. R.; Knowles, R. R. Light-driven depolymerization of native lignin enabled by proton-coupled electron transfer. ACS Catal. 2020, 10 (1), 800-805.
(18) Wu, X.; Fan, X.; Xie, S.; Lin, J.; Cheng, J.; Zhang, Q.; Chen, L.; Wang, Y Solar energy-driven lignin-first approach to full utilization of lignocellulosic biomass under mild conditions. Nat. Catal. 2018, 1 (10), 772-780.
(19) Wu, X.; Xie, S.; Liu, C.; Zhou, C.; Lin, J.; Kang, J.; Zhang, Q.; Wang, Z.; Wang, Y. Ligand-controlled photocatalysis of CdS quantum dots for lignin valorization under visible light. ACS Catal. 2019, 9 (9), 8443-8451.
(20) Chen, K.; Schwarz, J.; Karl, T. A.; Chatterjee, A.; König, B. Visible light induced redox neutral fragmentation of 1,2-diol derivatives. Chem. Commun. 2019, 55 (87), 13144-13147.
(21) Zhu, Q.; Nocera, D. G. Catalytic C (β)-O Bond Cleavage of Lignin in a One-Step Reaction Enabled by a Spin-Center Shift. ACS Catal. 2021, 11 (22), 14181-14187.
(22) Bosque, I.; Magallanes, G.; Rigoulet, M.; Karkss, M. D.; Stephenson, C. R. Redox catalysis facilitates lignin depolymerization. ACS Cent. Sci. 2017, 3 (6), 621-628.
(23) Yang, C.; Karkss, M. D.; Magallanes, G.; Chan, K.; Stephenson, C. R. Organocatalytic approach to photochemical lignin fragmentation. Org. Lett. 2020, 22 (20), 8082-8085.
(24) Li, S.; Kim, S.; Davis, A. H.; Zhuang, J.; Shuler, E. W.; Willinger, D.; Lee, J.-J.; Zheng, W.; Sherman, B. D.; Yoo, C. G.; et al. Photocatalytic Chemoselective C—C Bond Cleavage at Room Temperature in Dye-Sensitized Photoelectrochemical Cells. ACS Catal. 2021, 11 (7), 3771-3781.
(25) Li, S.; Wijethunga, U. K.; Davis, A. H.; Kim, S.; Zheng, W.; Sherman, B. D.; Yoo, C. G.; Leem, G. Ru (II) Polypyridyl-Modified TiO2 Nanoparticles for Photocatalytic C—C/C—O Bond Cleavage at Room Temperature. ACS Appl. Nano Mater. 2022, 5 (1), 948-956.
(26) Liu, G.; Jin, C.; Huo, S.; Kong, Z.; Chu, F. Preparation and properties of novel bio-based epoxy resin thermosets from lignin oligomers and cardanol. Int. J. Biol. Macromol. 2021, 193, 1400-1408.
(27) Van Aelst, K.; Van Sinay, E.; Vangeel, T.; Zhang, Y; Renders, T.; Van den Bosch, S.; Van Aelst, J.; Sels, B. F. Low molecular weight and highly functional RCF lignin products as a full bisphenol a replacer in bio-based epoxy resins. Chem. Commun. 2021, 57 (46), 5642-5645.
(28) Vendamme, R.; Behaghel de Bueren, J.; Gracia-Vitoria, J.; Isnard, F.; Mulunda, M. M.; Ortiz, P.; Wadekar, M.; Vanbroekhoven, K.; Wegmann, C.; Buser, R.; et al. Aldehyde-assisted lignocellulose fractionation provides unique lignin oligomers for the design of tunable polyurethane bioresins. Biomacromolecules 2020, 21 (10), 4135-4148.
(29) Gioia, C.; Lo Re, G.; Lawoko, M.; Berglund, L. Tunable thermosetting epoxies based on fractionated and well-characterized lignins. J. Am. Chem. Soc. 2018, 140 (11), 4054-4061.
(30) Cui, M.; Nguyen, N. A.; Bonnesen, P. V.; Uhrig, D.; Keum, J. K.; Naskar, A. K. Rigid oligomer from lignin in designing of tough, self-healing elastomers. ACS Macro Lett. 2018, 7 (11), 1328-1332.
(31) Moreno, A.; Morsali, M.; Sipponen, M. H. Catalyst-Free Synthesis of Lignin Vitrimers with Tunable Mechanical Properties: Circular Polymers and Recoverable Adhesives. ACS Appl. Mater. Interfaces 2021, 13 (48), 57952-57961.
(32) Tzirakis, M. D.; Lykakis, I. N.; Orfanopoulos, M. Decatungstate as an efficient photocatalyst in organic chemistry. Chem. Soc. Rev. 2009, 38 (9), 2609-2621.
(33) Wan, T.; Wen, Z.; Laudadio, G.; Capaldo, L.; Lammers, R.; Rincön, J. A.; Garcia-Losada, P.; Mateos, C.; Frederick, M. O.; Broersma, R.; et al. Accelerated and scalable C (sp3)-H amination via decatungstate photocatalysis using a flow photoreactor equipped with high-intensity LEDs. ACS Cent. Sci. 2022, 8 (1), 51-56.
(34) Laudadio, G.; Deng, Y.; van der Wal, K.; Ravelli, D.; Nuno, M.; Fagnoni, M.; Guthrie, D.; Sun, Y.; Noel, T. C (sp3)-H functionalizations of light hydrocarbons using decatungstate photo-catalysis in flow. Science 2020, 369 (6499), 92-96.
(35) Sarver, P. J.; Bacauanu, V.; Schultz, D. M.; DiRocco, D. A.; Lam, Y.-h.; Sherer, E. C.; MacMillan, D. W. The merger of decatungstate and copper catalysis to enable aliphatic C (sp3)-H trifluoromethylation. Nat. Chem. 2020, 12 (5), 459-467.
(36) Sarver, P. J.; Bissonnette, N. B.; MacMillan, D. W. Decatungstate-Catalyzed C (sp 3)-H Sulfinylation: Rapid Access to Diverse Organosulfur Functionality. J. Am. Chem. Soc. 2021, 143 (26), 9737-9743.
(37) Laudadio, G.; Govaerts, S.; Wang, Y.; Ravelli, D.; Koolman, H. F.; Fagnoni, M.; Djuric, S. W.; Noel, T. Selective C (sp3)-H aerobic oxidation enabled by decatungstate photocatalysis in flow. Angew. Chem. 2018, 130 (15), 4142-4146.
(38) Xia, P.; Raulerson, E. K.; Coleman, D.; Gerke, C. S.; Mangolini, L.; Tang, M. L.; Roberts, S. T. Achieving spin-triplet exciton transfer between silicon and molecular acceptors for photon upconversion. Nat. Chem. 2020, 12 (2), 137-144.
(39) Hu, A.; Chen, Y; Guo, J.; Yu, N.; An, Q.; Zuo, Z. CeriumCatalyzed Formal Cycloaddition of Cycloalkanols with Alkenes through Dual Photoexcitation. J. Am. Chem. Soc. 2018, 140 (42), 13580-1358.
(40) Deng, W.; Zhang, H.; Wu, X.; Li, R.; Zhang, Q.; Wang, Y. Oxidative conversion of lignin and lignin model compounds catalyzed by CeO 2-supported Pd nanoparticles. Green Chem. 2015, 17 (11), 5009-5018.
(41) Zhou, Y.; Klinger, G. E.; Hegg, E. L.; Saffron, C. M.; Jackson, J. E. Multiple Mechanisms Mapped in Aryl Alkyl Ether Cleavage via Aqueous Electrocatalytic Hydrogenation over Skeletal Nickel. J. Am. Chem. Soc. 2020, 142 (8), 4037-4050.
(42) Chao, A.; Negulescu, I.; Zhang, D. Dynamic Covalent Polymer Networks Based on Degenerative Imine Bond Exchange: Tuning the Malleability and Self-Healing Properties by Solvent. Macromolecules 2016, 49 (17), 6277-6284.
(43) Belowich, M. E.; Stoddart, J. F. Dynamic imine chemistry. Chem. Soc. Rev. 2012, 41 (6), 2003-2024.
(44) Snyder, R. L.; Lidston, C. A. L.; De Hoe, G. X.; Parvulescu, M. J. S.; Hillmyer, M. A.; Coates, G. W. Mechanically robust and reprocessable imine exchange networks from modular polyester prepolymers. Polym. Chem. 2020, 11 (33), 5346-5355.
(45) Denissen, W.; Droesbeke, M.; Nicolay, R.; Leibler, L.; Winne, J. M.; Du Prez, F. E. Chemical control of the viscoelastic properties of vinylogous urethane vitrimers. Nat. Commun. 2017, 8 (1), 14857.
(46) Denissen, W.; Rivero, G.; Nicolay, R.; Leibler, L.; Winne, J. M.; Du Prez, F. E. Vinylogous Urethane Vitrimers. Adv. Funct. Mater. 2015, 25 (16), 2451-2457.
(47) Zijlstra, D. S.; de Santi, A.; Oldenburger, B.; de Vries, J.; Barta, K.; Deuss, P. J. Extraction of lignin with high β-O-4 content by mild ethanol extraction and its effect on the depolymerization yield. J. Vis. Exp. 2019, No. 143, No. e58575.
(48) Wen, J.-L.; Sun, S.-L.; Yuan, T.-Q.; Xu, F.; Sun, R.-C. Structural elucidation of lignin polymers of Eucalyptus chips during organosolv pretreatment and extended delignification. J. Agric. Food Chem. 2013, 61 (46), 11067-11075.
(49) Wen, J.-L.; Sun, S.-L.; Xue, B.-L.; Sun, R.-C. Recent Advances in Characterization of Lignin Polymer by Solution-State Nuclear Magnetic Resonance (NMR) Methodology. Materials 2013, 6 (1), 359-391.
(50) Lessard, J. J.; Garcia, L. F.; Easterling, C. P.; Sims, M. B.; Bentz, K. C.; Arencibia, S.; Savin, D. A.; Sumerlin, B. S. Catalyst-Free Vitrimers from Vinyl Polymers. Macromolecules 2019, 52 (5), 2105-2111.
(51) Lessard, J. J.; Scheutz, G. M.; Sung, S. H.; Lantz, K. A.; Epps, T. H.; Sumerlin, B. S. Block Copolymer Vitrimers. J. Am. Chem. Soc. 2020, 142 (1), 283-289.
(52) Abu-Omar, M. M.; Barta, K.; Beckham, G. T.; Luterbacher, J. S.; Ralph, J.; Rinaldi, R.; Romen-Leshkov, Y; Samec, J. S.; Sels, B. F.; Wang, F., Guidelines for performing lignin-first biorefining. Energy & Environmental Science 2021, 14 (1), 262-292.
(53) Constant, S.; Lancefield, C. S.; Weckhuysen, B. M.; Bruijnincx, P. C., Quantification and classification of carbonyls in industrial humins and lignins by 19F NMR. ACS Sustainable Chemistry & Engineering 2017, 5 (1), 965-972.
(54) Protti, S.; Ravelli, D.; Fagnoni, M.; Albini, A., Solar light-driven photocatalyzed alkylations. Chemistry on the window ledge. Chemical communications 2009, (47), 7351-7353.
(55) Zijlstra, D. S.; de Santi, A.; Oldenburger, B.; de Vries, J.; Barta, K.; Deuss, P. J., Extraction of lignin with high β-O-4 content by mild ethanol extraction and its effect on the depolymerization yield. JoVE (Journal of Visualized Experiments) 2019, (143), e58575.
(56) Wu, X.; Fan, X.; Xie, S.; Lin, J.; Cheng, J.; Zhang, Q.; Chen, L.; Wang, Y., Solar energy-driven lignin-first approach to full utilization of lignocellulosic biomass under mild conditions. Nature catalysis 2018, 1 (10), 772-780.
(57) Lessard, J. J.; Garcia, L. F.; Easterling, C. P.; Sims, M. B.; Bentz, K. C.; Arencibia, S.; Savin, D. A.; Sumerlin, B. S., Catalyst-Free Vitrimers from Vinyl Polymers. Macromolecules 2019, 52 (5), 2105-2111.

We claim:

1. A method for selective partial depolymerization of natural lignin source, the method comprising the steps of:
(1) obtaining a natural lignin source;
(2) performing a photocatalytic activation of a β-O-4 bond in the natural lignin source by using a photocatalytic activation system comprising a catalyst comprising tetrabutylammonium decatungstate (TBADT) and a light source with light in an ultraviolet region shined on the natural lignin source; and
(3) adding an exogenous electron mediator/scavenger system comprising an exogenous electron mediator to promote a cleavage or a bond-scission of the β-O-4 bond and/or an exogenous electron scavenger to promote an oxidation of the β-O-4 bond;
whereby the addition of the exogenous electron mediator increases a yield of an oligomer but decreases an amount of carbonyl (C═O) groups in the oligomer and the addition of the exogenous electron scavenger increases the amount of carbonyl (C═O) groups in the oligomer and slightly changes the yield of the oligomer, wherein the yield of the oligomer is a weight of the oligomer divided by a weight of the natural lignin source.

2. The method of claim 1, wherein the natural lignin source is selected from a group consisting of hardwood, softwood, annual fibers, lignocellulosic biomass, wheat straw, rice straw, switchgrass, miscanthus, poplar wood, pine wood, corn stover, and bagasse.

3. The method of claim 1, wherein the photocatalytic activation system further comprises at least one of a solvent, a pH modifier, and a surfactant.

4. The method of claim 3, wherein the solvent is selected from a group consisting of water, acetonitrile, dimethylformamide, ethanol, acetone (AC), dichloromethane (DCM), dioxane, ethyl acetate (EA), hexane (Hex), methanol (MeOH), n-butylamine, and tetrahydrofuran (THF).

5. The method of claim 3, wherein the pH modifier is selected from a group consisting of an acid and a base, wherein the acid is selected from a group consisting of hydrochloric acid, sulfuric acid, phosphoric acid, and acetic acid, and wherein the base is selected from a group consisting of sodium hydroxide, potassium hydroxide, and ammonium hydroxide.

6. The method of claim 3, wherein the surfactant is selected from a group consisting of sodium dodecyl sulfate, cetyltrimethylammonium bromide, and Triton X-100.

7. The method of claim 1, wherein the light source emits light with a wavelength ranging from about 200 nm to about 400 nm.

8. The method of claim 1, wherein the exogenous electron mediator is selected from a group consisting of methyl viologen, benzyl viologen, 2,2′-bipyridine, and 9,10-diphenylanthracene (DPA).

9. The method of claim 1, wherein the exogenous electron scavenger is selected from a group consisting of oxygen, hydrogen peroxide, and ammonium persulfate.

10. The method of claim 1, wherein the oligomer has a number average molecular weight ranging from about 500 Da to about 10000 Da.

11. The method of claim 1, wherein the oligomer has a dispersity ranging from about 1.5 to about 3.5.

12. The method of claim 1, wherein the oligomer has a yield ranging from about 10 wt % to about 90 wt % based on the weight of the natural lignin source.

13. The method of claim 1, wherein the oligomer has an amount of carbonyl (C═O) groups ranging from about 0.1 mmol/g to about 5 mmol/g.

14. The method of claim 1, wherein the oligomer is further converted to a chemically recyclable polymer network.

15. The method of claim 14, wherein the chemically recyclable polymer network is selected from a group consisting of a polyurethane, a polyester, a polyamide, and a polycarbonate.

16. The method of claim 1, wherein the photocatalytic activation and the addition of the exogenous electron mediator/scavenger system are performed simultaneously or sequentially.

17. The method of claim 1, wherein the photocatalytic activation is performed at a temperature ranging from about 10° C. to about 150° C.

18. The method of claim 1, wherein the photocatalytic activation is performed for a duration ranging from about 1 hour to about 24 hours.

19. The method of claim 1, wherein the exogenous electron mediator/scavenger system is added in an amount ranging from about 0.1 mol % to about 10 mol % based on the moles of the β-O-4 bond in the natural lignin source.

20. The method of claim 1, wherein without added electron scavengers, TBADT is capable of extracting electrons and protons from the natural lignin; the TBADT acting as an oxidant, which will produce an amount of oxidation products (19.0%) even under a pure nitrogen atmosphere.
